(19) United States
(12) Patent Application Publication
(10) Pub. No.: US 2012/0207713 A1
(43) Pub. Date: Aug. 16, 2012

(54) PROBIOTIC BIFIDOBACTERIUM STRAINS

(75) Inventors: John MacSharry, Cork (IE); Liam O'Mahony, County Cork (IE); David O'Sullivan, County Cork (IE); Barry Kiely, County Cork (IE)

(73) Assignee: ALIMENTARY HEALTH LIMITED, Cork (IE)

(21) Appl. No.: 13/347,289

(22) Filed: Jan. 10, 2012

Related U.S. Application Data
(63) Continuation-in-part of application No. 12/450,427, filed on Apr. 26, 2010.
(60) Provisional application No. 61/457,130, filed on Jan. 10, 2011.
(60) Provisional application No. 60/907,310, filed on Mar. 28, 2007.

(51) Int. Cl. A61K 35/74; A61P 1/04; A61P 35/00; A61P 37/00; A61P 19/02; A61P 29/00; A61P 1/12; A61P 25/28; A61P 3/10; A61P 31/18; A61P 25/00; A61P 31/04; A61P 31/12; A61P 31/10; A61P 37/06; A61P 11/06; A61P 3/04; A61P 27/14; A61P 35/04; A61P 9/00; A61P 13/12; A61P 1/16; A61P 7/06; A61P 3/02; A61P 19/10; A61P 5/00; A61P 17/02; A61P 17/00; A61P 17/06; A61P 17/10; A61P 37/04; C12N 1/20; A61P 1/02; A61P 13/00; A61P 15/00; A61P 1/00

(52) U.S. Cl. 424/93.4; 435/252.1

(57) ABSTRACT

Bifidobacterium strain AH1206 or mutants or variants thereof are immunomodulatory following oral consumption and are useful in the prophylaxis and/or treatment of inflammatory activity for example undesirable gastrointestinal inflammatory activity such as inflammatory bowel disease.

The present application claims the benefit of U.S. Provisional Application No. 61/457,130 filed Jan. 10, 2011, and is a continuation-in-part of U.S. patent application Ser. No. 12/450,427 filed Sep. 25, 2009, which is a nationalisation of PCT/IE2008/000033 filed on Mar. 28, 2008, which claims the benefit of U.S. Provisional Application No. 60/907,310 filed Mar. 28, 2007, the disclosures of which are incorporated herein by reference.

### INTRODUCTION

The invention relates to a Bifidobacterium strain and its use as a probiotic bacteria in particular as an immunomodulatory biotherapeutic agent.

The defense mechanisms to protect the human gastrointestinal tract from colonization by intestinal bacteria are highly complex and involve both immunological and non-immunological aspects (1). Innate defense mechanisms include the low pH of the stomach, bile salts, peristalsis, mucin layers and anti-microbial compounds such as lysozyme (2). Immunological mechanisms include specialized lymphoid aggregates, underlying M cells, called peyers patches which are distributed throughout the small intestine and colon (3). Luminal antigens presented at these sites result in stimulation of appropriate T and B cell subsets with establishment of cytokine networks and secretion of antibodies into the gastrointestinal tract (4). In addition, antigen presentation may occur via epithelial cells to intraepithelial lymphocytes and to the underlying lamina propria immune cells (5). Therefore, the host invests substantially in immunological defense of the gastrointestinal tract. However, as the gastrointestinal mucosa is the largest surface at which the host interacts with the external environment, specific control mechanisms must be in place to regulate immune responsiveness to the 100 tons of food which is handled by the gastrointestinal tract over an average lifetime. Furthermore, the gut is colonized by over 500 species of bacteria numbering 10¹¹-10¹²/g in the colon. Thus, these control mechanisms must be capable of distinguishing non-pathogenic adherent bacteria from invasive pathogens, which would cause significant damage to the host. In fact, the intestinal flora contributes to defense of the host by competing with newly ingested potentially pathogenic micro-organisms.

Bacteria present in the human gastrointestinal tract can promote inflammation. Aberrant immune responses to the indigenous microflora have been implicated in certain disease states, such as inflammatory bowel disease. Antigens associated with the normal flora usually lead to immunological tolerance and failure to achieve this tolerance is a major mechanism of mucosal inflammation (6). Evidence for this breakdown in tolerance includes an increase in antibody levels directed against the gut flora in patients with inflammatory bowel syndrome (IBD).

The present invention is directed towards a Bifidobacterium strain which has been shown to have immunomodulatory effects, by modulating cytokine levels or by antagonizing and excluding pro-inflammatory micro-organisms from the gastrointestinal tract.

### STATEMENTS OF INVENTION

According to the invention there is provided Bifidobacterium strain AH1206 (NCIMB 41382) or mutants or variants thereof.

The mutant may be a genetically modified mutant. The variant may be a naturally occurring variant of Bifidobacterium.

The strain may be a probiotic. It may be in the form of a biologically pure culture.

The invention also provides an isolated strain of Bifidobacterium NCIMB 41382.

In one embodiment of the invention Bifidobacterium strains are in the form of viable cells. Alternatively Bifidobacterium strains are in the form of non-viable cells.

In one embodiment of the invention the Bifidobacterium strains are isolated from infant faeces, the Bifidobacterium strains being significantly immunomodulatory following oral consumption in humans.

The invention also provides a formulation which comprises the Bifidobacterium strain of the invention.

In one embodiment of the invention the formulation includes another probiotic material.

In one embodiment of the invention the formulation includes a prebiotic material.

Preferably the formulation includes an ingestable carrier. The ingestable carrier may be a pharmaceutically acceptable carrier such as a capsule, tablet or powder. Preferably the ingestable carrier is a food product such as acidified milk, yoghurt, frozen yoghurt, milk powder, milk concentrate, cheese spreads, dressings or beverages.

In one embodiment of the invention the formulation of the invention further comprises a protein and/or peptide, in particular proteins and/or peptides that are rich in glutamine/glutamate, a lipid, a carbohydrate, a vitamin, mineral and/or trace element.

In one embodiment of the invention the Bifidobacterium strain is present in the formulation at more than 10⁶ cfu per gram of delivery system. Preferably the formulation includes any one or more of an adjuvant, a bacterial component, a drug entity or a biological compound.

In one embodiment of the invention the formulation is for immunisation and vaccination protocols.

The invention further provides a Bifidobacterium strain or a formulation of the invention for use as foodstuffs, as a medicament, for use in the prophylaxis and/or treatment of undesirable inflammatory activity, for use in the prophylaxis and/or treatment of undesirable respiratory inflammatory activity such as asthma, for use in the prophylaxis and/or treatment of undesirable gastrointestinal inflammatory activity such as inflammatory bowel disease eg. Crohns disease or ulcerative colitis, irritable bowel syndrome, pouchitis, or post infection colitis, for use in the prophylaxis and/or treatment of gastrointestinal cancer(s), for use in the prophylaxis and/or treatment of systemic disease such as rheumatoid arthritis, for use in the prophylaxis and/or treatment of autoimmune disorders due to undesirable inflammatory activity, for use in the prophylaxis and/or treatment of cancer due to undesirable inflammatory activity, for use in the prophylaxis of cancer, for use in the prophylaxis and/or treatment of diarrhoeal disease due to undesirable inflammatory activity, such as Clostridium difficile associated diarrhoea, Rotavirus associated diarrhoea or post infective diarrhoea, for use in the prophylaxis and/or treatment of diarrhoeal disease due to an infectious agent, such as E. coli.

The invention also provides a Bifidobacterium strain or a formulation of the invention for use in the preparation of an anti-inflammatory biotherapeutic agent for the prophylaxis and/or treatment of undesirable inflammatory activity or for use in the preparation of anti-inflammatory biotherapeutic agents for the prophylaxis and/or treatment of undesirable inflammatory activity.

In one embodiment of the invention the strain of the invention act by antagonising and excluding proinflammatory micro-organisms from the gastrointestinal tract.

The invention also provides a Bifidobacterium strain or a formulation of the invention for use in the preparation of anti-inflammatory biotherapeutic agents for reducing the levels of pro-inflammatory cytokines.

The invention further provides a Bifidobacterium strain for use in the preparation of anti-inflammatory biotherapeutic agents for modifying the levels of IL-10.

The invention may also provides for the use of a Bifidobacterium strain as a anti-infective probiotic due to their ability to antagonise the growth of pathogenic species.

The invention may also provide for the use of a Bifidobacterium strain in the preparation of a medicament for treating asthma and/or allergy. The medicament may be in a form suitable for inhalation.

The invention may further provide for the use of a Bifidobacterium strain in the preparation of anti-inflammatory biotherapeutic agents for reducing levels of IgE.

We have found that particular strains of Bifidobacterium elicit immunomodulatory effects in vitro.

The invention may therefore have potential therapeutic value in the prophylaxis or treatment of dysregulated immune responses, such as undesirable inflammatory reactions for example asthma and/or allergy.

Bifidobacterium are commensal microorganisms. They have been isolated from the microbial flora within the human gastrointestinal tract. The immune system within the gastrointestinal tract cannot have a pronounced reaction to members of this flora, as the resulting inflammatory activity would also destroy host cells and tissue function. Therefore, some mechanism(s) exist whereby the immune system can recognize commensal non-pathogenic members of the gastrointestinal flora as being different to pathogenic organisms. This ensures that damage to host tissues is restricted and a defensive barrier is still maintained.

A deposit of Bifidobacterium strain AH1205 was made at the National Collections of Industrial and Marine Bacteria Limited (NCIMB) on May 11, 2006 and accorded the accession number NCIMB 41387.

A deposit of Bifidobacterium longum strain AH1206 was made at the National Collections of Industrial and Marine Bacteria Limited (NCIMB) on Mar. 15, 2006 and accorded the accession number NCIMB 41382.

A deposit of Lactobacillus salivarius strain AH102 was made at the National Collections of Industrial and Marine Bacteria Limited (NCIMB) on Apr. 20, 2000 and accorded the accession number NCIMB 41044.

The Bifidobacterium longum may be a genetically modified mutant or it may be a naturally occurring variant thereof.

Preferably the Bifidobacterium longum is in the form of viable cells.

Alternatively the Bifidobacterium longum may be in the form of non-viable cells.

It will be appreciated that the specific Bifidobacterium strain of the invention may be administered to animals (including humans) in an orally ingestible form in a conventional preparation such as capsules, microcapsules, tablets, granules, powder, troches, pills, suppositories, suspensions and syrups. Suitable formulations may be prepared by methods commonly employed using conventional organic and inorganic additives. The amount of active ingredient in the medical composition may be at a level that will exercise the desired therapeutic effect.

The formulation may also include a bacterial component, a drug entity or a biological compound.

In addition a vaccine comprising the strains of the invention may be prepared using any suitable known method and may include a pharmaceutically acceptable carrier or adjuvant.

Throughout the specification the terms mutant, variant and genetically modified mutant include a strain of Bifidobacteria whose genetic and/or phenotypic properties are altered compared to the parent strain. Naturally occurring variant of Bifidobacterium longum includes the spontaneous alterations of targeted properties selectively isolated. Deliberate alteration of parent strain properties is accomplished by conventional (in vitro) genetic manipulation technologies, such as gene disruption, conjugative transfer, etc. Genetic modification includes introduction of exogenous and/or endogenous DNA sequences into the genome of a Bifidobacteria strain, for example by insertion into the genome of the bacterial strain by vectors, including plasmid DNA, or bacteriophages.

Natural or induced mutations include at least single base alterations such as deletion, insertion, tansversion or other DNA modifications which may result in alteration of the amino acid sequence encoded by the DNA sequence.

The terms mutant, variant and genetically modified mutant also include a strain of Bifidobacteria that has undergone genetic alterations that accumulate in a genome at a rate which is consistent in nature for all micro-organisms and/or genetic alterations which occur through spontaneous mutation and/or acquisition of genes and/or loss of genes which is not achieved by deliberate (in vitro) manipulation of the genome but is achieved through the natural selection of variants and/or mutants that provide a selective advantage to support the survival of the bacterium when exposed to environmental pressures such as antibiotics. A mutant can be created by the deliberate (in vitro) insertion of specific genes into the genome which do not fundamentally alter the biochemical functionality of the organism but whose products can be used for identification or selection of the bacterium, for example antibiotic resistance.

A person skilled in the art would appreciate that mutant or variant strains of Bifidobacteria can be identified by DNA sequence homology analysis with the parent strain. Strains of Bifidobacteria having a close sequence identity with the parent strain are considered to be mutant or variant strains. A Bifidobacteria strain with a sequence identity (homology) of 96% or more, such as 97% or more or 98% or more or 99% or more with the parent DNA sequence may be considered to be a mutant or variant. Sequence homology may be determined using on-line homology algorithm “BLAST” program, publicly available at http://www.ncbi.nlm.nih,gov/BLAST/.

Mutants of the parent strain also include derived Bifidobacteria strains having at least 85% sequence homology such as at least 90% sequence homology of at least 95% sequence homology to the 16 s-23 s intergenic spacer polynucleotide sequence of the parent strain. These mutants may further comprise DNA mutations in other DNA sequences in the bacterial genome.

### BRIEF DESCRIPTION OF THE DRAWINGS

FIG. 1 is a BOX PCR (bioanalyser) barcode profile for B. longum AH1206. Base pair sizes were determined using the Agilent 2100 software;

FIG. 2 is a graph illustrating the faecal recovery of B. longum AH1206 over an 8 day feeding period and demonstrates that AH1206 can survive the murine gastrointestinal tract;

FIG. 3 is a bar graph showing the effect of B. longum AH1206 on IL-10 cytokine production by human PBMCs. Results are expressed as mean+/−SE (n=6);

FIG. 4 is a bar graph showing the effect of B. longum AH1206 feeding on eosinophil recruitment to the lungs of sensitized mice. (A) total number of cells present in bronchoalveolar lavage (BAL) were reduced in AH1206 fed mice; (B) Differential cell counts on BAL revealed that the reduction in cell numbers was primarily in the eosinophil population. (Cell number is expressed on the y-axis (×10⁴); *p<0.05 versus placebo);

FIGS. 5 A and B are graphs showing the effect of probiotic bacterial strain AH1206 (A) and placebo (B) on total cell numbers in bronchoalveolar lavage fluid following ovalbumin (OVA) challenge in sensitised animals (n=10/group, *=p<0.05 compared to OVA challenge alone);

FIGS. 6 A and B are graphs showing the effect of probiotic bacterial strain AH1206 (A) and placebo (B) treatment on airway responsiveness to methacholine, as assessed by changes in enhanced pause (Penh) in ovalbumin (OVA)-sensitised mice 24 hours after intranasal challenge with OVA or saline. Each data point represents the mean±SEM (n=10/groups*p=<0.05 compared to OVA alone);

FIG. 7 is a graph showing the TNF cytokine level in bronchoalveolar lavage (BAL) fluid from ovalbumin (OVA)-sensitised mice. Each column represents the mean±SEM (n=10, *p<0.05 compared to OVA challenged, MRS broth treated control);

FIGS. 8 A and B are graphs showing the effect of oral treatment with probiotic strain AH1206 an TNF (A) and IFNγ (B) cytokine production from activated splenocytes isolated from OVA-sensitised mice (CD3/CD28 stimulated splenocytes). Each column represents the mean±SEM (n=10, *p=<0.05 compared to OVA challenge, MRS broth treated control);

FIG. 9 is a graph showing that the levels of OVA-specific IgE in serum isolated from mice fed AH1206 probiotic bacteria was significantly lower than the non-probiotic fed controls (**p=<0.01);

FIG. 10 is a graph illustrating the effect of oral treatment of probiotic strain AH1206 on TNF a production from activated splenocytes isolated from OVA-sensitised mice (CD3/CD28 stimulated splenocytes). The mean is illustrated for each group (*p=<0.05, **p=<0.01 compared to OVA and CT challenge, MRS broth treated control);

FIG. 11 is a graph illustrating that CD4+CD25+ cells from AH1206 fed animals substantially reduced proliferation (n=10 for all groups except the control, in which n=20);

FIGS. 12 A and B are graphs showing the percentage of cells in the CD4+ population that are also CD25+, as assessed by flow cytometry (n=11 for the unfed group, n=20 for placebo group, and n=10 for the AH1206 fed group);

FIG. 13. The percentage of CD4/CD25+ cells expressing the transcription factor Foxp3 is significantly upregulated in germ free mice consuming AH1206 (n=8 or 9 per group). *p<0.05 vs placebo;

FIG. 14 is a graph illustrating the stability of probiotic strain AH1206 over 3 months;

FIG. 15 Murine study outline. The study duration and age of the animals in each study group is illustrated;

FIG. 16 Bifidobacterium AH1206 consumption induces T regulatory cells in murine models. The bacterial strains Bifidobacterium AH1205, Bifidobacterium AH1206 or Lactobacillus AH102 were consumed by mice in three separate studies. Immediately following birth, mice consumed each strain for 8 weeks (A) or mice were administered each strain commencing at 8 weeks of age (B). CD4/CD25+ T cells were increased in both experimental settings only for the Bifidobacterium AH1206-fed mice. In addition, germ-free mice consumed Bifidobacterium AH1205 or Bifidobacterium AH1206 for 3 weeks, beginning at 8 weeks of age (C). A significant increase in splenic CD4/CD25/Foxp3+ cells was observed only in Bifidobacterium AH1206-fed animals. Isolated CD4/CD25+ T cells from adult wild-type mice exhibited a suppressive phenotype as determined by measuring the proliferative response of CFSE-labelled responder CD4 T cells (D). Results are expressed as the mean+/−SE for each group (n=8-10 animals per group).*p<0.05 versus placebo;

FIG. 17 Microbes transit the gastrointestinal tract at high numbers. The bacterial strains Bifidobacterium AH1205, Bifidobacterium AH1206 or Lactobacillus AH102 were consumed by conventionally colonised animals and all three commensal strains transited the murine gut at similar levels over an 8 day feeding period (A). In addition, both Bifidobacterium AH1205 and Bifidobacterium AH1206 transited the germ-free gut at high numbers. Results are expressed as the mean+/−SE for each group (n=3-5 animals per group);

FIG. 18 Bifidobacterium AH1206 reduces respiratory inflammation following OVA administration. Bifidobacterium AH1205, Bifidobacterium AH1206 or Lactobacillus AH102 were consumed by mice during sensitisation and challenge with OVA. The inflammatory cell influx to the lung (both total cell count and eosinophils) was significantly reduced in Bifidobacterium AH1206-fed animals (A). In addition, bronchoalveolar lavage TNF-α levels were significantly reduced in Bifidobacterium AH1205 and Bifidobacterium AH1206-fed mice (B). Results are expressed as the mean+/−SE for each group (n>5 animals per group).*p<0.05 versus placebo;

FIG. 19 Bifidobacterium AH1206 reduces serum OVA-IgE levels. Bifidobacterium AH1205, Bifidobacterium AH1206 or Lactobacillus AH102 were consumed by mice during oral sensitisation with OVA and cholera toxin (CT). Treatment with OVA-CT alone significantly increased the serum IgE level to OVA while co-feeding with Bifidobacterium AH1206 blocked the increase in IgE levels. Results are expressed as the serum OVA-IgE level for each animal tested (n>4 animals per group). *p<0.05 versus placebo; and

FIG. 20 Bifidobacterium AH1206 reduces CD3/CD28-stimulated cytokine production. Splenocytes from Bifidobacterium AH1206 and Lactobacillus AH102-fed animals were isolated following OVA-CT sensitisation and stimulated in vitro with anti-CD3 and anti-CD28 antibodies. TNF-α (A), IL-4 (B), IL-2 (C) and IFN-γ (D) levels in splenocyte culture supernatants were significantly reduced for the Bifidobacterium AH1206-fed animals. Results are expressed as the cytokine level for each animal tested (n≧5 animals per group). *p<0.05 versus placebo.

### DETAILED DESCRIPTION

We have found that Bifidobacterium longum strain AH1206 is not only acid and bile tolerant and transits the gastrointestinal tracts but also, surprisingly has immunomodulatory effects, by modulating cytokine levels or by antagonising and excluding pro-inflammatory or immunomodulatory micro-organisms from the gastrointestinal tract. Indeed, consumption of B. longum AH1206 significantly reduces recruitment of disease causing cells to the lungs of a murine asthma model.

The general use of probiotic bacteria is in the form of viable cells. However, it can also be extended to non-viable cells such as killed cultures or compositions containing beneficial factors expressed by the probiotic bacteria. This could include thermally killed micro-organisms or micro-organisms killed by exposure to altered pH or subjection to pressure. With non-viable cells product preparation is simpler, cells may be incorporated easily into pharmaceuticals and storage requirements are much less limited than viable cells. Lactobacillus casei YIT 9018 offers an example of the effective use of heat killed cells as a method for the treatment and/or prevention of tumour growth as described in U.S. Pat. No. 4,347,240.

It is unknown whether intact bacteria are required to exert an immunomodulatory effect or if individual active components of the invention can be utilized alone. Proinflammatory components of certain bacterial strains have been identified. The proinflammatory effects of gram-negative bacteria are mediated by lipopolysaccharide (LPS). LPS alone induces a proinflammatory network, partially due to LPS binding to the CD14 receptor on monocytes. It is assumed that components of probiotic bacteria possess immunomodulatory activity, due to the effects of the whole cell. Upon isolation of these components, pharmaceutical grade manipulation is anticipated.

IL-10 is produced by T cells, B cells, monocytes and macrophages. This cytokine augments the proliferation and differentiation of B cells into antibody secreting cells. IL-10 exhibits mostly anti-inflammatory activities. It up-regulates IL-1RA expression by monocytes and suppresses the majority of monocyte inflammatory activities. IL-10 inhibits monocyte production of cytokines, reactive oxygen and nitrogen intermediates, MHC class II expression, parasite killing and IL-10 production via a feed back mechanism (7). This cytokine has also been shown to block monocyte production of intestinal collagenase and type IV collagenase by interfering with a PGE2-cAMP dependant pathway and therefore may be an important regulator of the connective tissue destruction seen in chronic inflammatory diseases.

The host response to infection is characterised by innate and acquired cellular and humoral immune reactions, designed to limit spread of the offending organism and to restore organ homeostasis. However, to limit the aggressiveness of collateral damage to host tissues, a range of regulatory constraints may be activated. Regulatory T cells (Tregs) serve one such mechanism. These are derived from the thymus but may also be induced in peripheral organs, including the gut mucosa. Deliberate administration of Treg cells suppresses inflammatory disease in a wide range of murine models including experimental autoimmune encephalomyelitis, inflammatory bowel disease, bacterial-induced colitis, collagen-induced arthritis, type I diabetes, airway osinophilic inflammation, graft-vs-host disease and organ transplantation. The forkhead transcription factor Foxp3 (forkhead box P3) is selectively expressed in Treg cells, is required for Treg development and function, and is sufficient to induce a Treg phenotype in conventional CD4 cells (19). Mutations in Foxp3 cause severe, multi-organ autoimmunity in both human and mouse. We have described a Bifidobacterium strain that generates CD25 positive/Foxp3 positive T regulatory cells in vivo.

The invention will be more clearly understood from the following examples.

### Example 1

### Characterisation of Bacteria Isolated from Infant Faeces. Demonstration of Probiotic Traits

### Isolation of Probiotic Bacteria

Fresh faeces was obtained from a 2 day old male breast fed infant and serially dilutions were plated on TPY (trypticase, peptone and yeast extract) and MRS (deMann, Rogosa and Sharpe) media supplemented with 0.05% cysteine and mupirocin. Plates were incubated in anaerobic jars (BBL, Oxoid) using CO₂ generating kits (Anaerocult A, Merck) for 2-5 days at 37° C. Gram positive, catalase negative rod-shaped or bifurcated/pleomorphic bacteria isolates were streaked for purity on to complex non-selective media (MRS and TPY). Isolates were routinely cultivated in MRS or TPY medium unless otherwise stated at 37° C. under anaerobic conditions. Presumptive Bifidobacterium were stocked in 40% glycerol and stored at −20° C. and −80° C.

Following isolation of a pure bifidobacteria strain, assigned the designation AH1206, microbiological characteristics were assessed and are summarized in Table 1 below. AH1206 is a gram positive, catalase negative pleomorphic shaped bacterium which is Fructose-6-Phoshate Phosphoketolase positive confirming its identity as a bifidobacterium. Using minimal media in which a single carbon source was inserted, AH1206 was able to grow on all carbon sources tested (Glucose, Lactose, Ribose, Arabinose, Galactose, Raffinose, Fructose, Malt Extract, Mannose, Maltose, Sucrose).

TABLE 1
Physiochemical characteristics of B. longum AH1206

| Strain Characteristics | B. longum AH1206 |
|---|---|
| Gram Stain | + |
| Catalase | − |
| Motility | − |
| F6PPK* | + |
| Milk coagulation | + |
| 45° C. anaerobic culture | − |
| 45° C. aerobic culture | − |
| CHO Fermentation: | |
| Glucose | + |
| Lactose | + |
| Ribose | + |
| Arabinose | + |
| Galactose | + |
| Raffinose | + |
| Fructose | + |
| Malt Extract | + |
| Mannose | + |
| Maltose | + |
| Sucrose | + |

*signifies Fructose-6-Phoshate Phosphoketolase Assay

### Species Identification

16 s Intergenic spacer (IGS) sequencing was performed to identify the species of bifidobacteria isolated. Briefly, DNA was isolated from AH1206 using 100 μl of Extraction Solution and 25 μA of Tissue Preparation solution (Sigma, XNAT2 Kit). The samples were incubated for 5 minutes at 95° C. and then 100 μl of Neutralization Solution (XNAT2 kit) was added. Genomic DNA solution was quantified using a Nanodrop spectrophotometer and stored at 4° C. PCR was performed using the IGS primers, IGS L: 5′-GCTGGATCACCTCCTTTC-3′ (SEQ ID No. 3) which is based on SEQ ID NO. 1 and IGS R: 5′-CTGGTGCCAAGGCATCCA-3′ (SEQ ID No. 4) which is based on SEQ ID NO. 2. The cycling conditions were 94° C. for 3 min (1 cycle), 94° C. for 30 sec, 53° C. for 30 sec, 72° C. for 30 sec (28 cycles). The PCR reaction contained 4 μl (50 ng) of DNA, PCR mix (XNAT2 kit), 0.4 μM IGS L and R primer (MWG Biotech, Germany). The PCR reactions were performed on an Eppendorf thermocycler. The PCR products (10 μl) were ran alongside a molecular weight marker (100 bp Ladder, Roche) on a 2% agarose EtBr stained gel in TAE, to determine the IGS profile. PCR products of Bifidobacterium (single band) were purified using the Promega Wizard PCR purification kit. The purified PCR products were sequenced using the primer sequences (above) for the intergenic spacer region. Sequence data was then searched against the NCBI nucleotide database to determine the identity of the strain by nucleotide homology. The resultant DNA sequence data was subjected to the NCBI standard nucleotide-to-nucleotide homology BLAST search engine (http://www.ncbi.nlm.nih.gov/BLAST/). The nearest match to the sequence was identified and then the sequences were aligned for comparison using DNASTAR MegAlign software. The sequences obtained can be viewed in the sequence listing in which SEQ ID NO. 1 is the IGS forward sequence and SEQ ID NO. 2 is the IGS reverse sequence. Searching the NCIMB database revealed that AH1206 has a unique IGS sequence with its closest sequence homology to a Bifidobacterium longum.

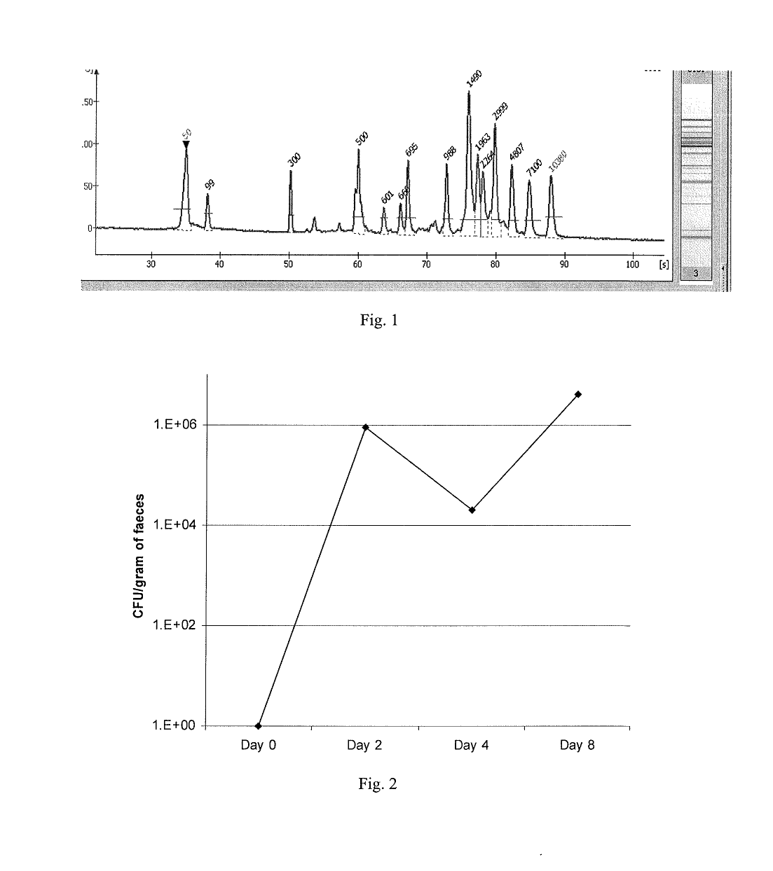

In order to develop a barcode PCR profile for AH1206, PCR was performed using BOX primers (8). The cycling conditions were 94° C. for 7 min (1 cycle); 94° C. for 1 minute, 65° C. for 8 minutes, (30 cycles) and 65° C. for 16 minutes. The PCR reaction contained 50 ng of DNA, PCR mix (XNAT2 kit) and 0.3 μM BOXA1R primer (5′-CTACGGCAAGGCGACGCTGACG-3′) (SEQ ID No. 5) (MWG Biotech, Germany). The PCR reactions were performed on an Eppendorf thermocycler. The PCR products (1 μl) were ran alongside a molecular weight marker (DNA 7500 ladder, Agilent, Germany) using the DNA 7500 LabChip® on the Agilent 2100 Bioanalyzer (Agilent, Germany). The barcode (PCR product profile) was determined using the Agilent Bioanalyzer software where peak number (PCR products) and size were identified (FIG. 1).

### Antibiotic Sensitivity Profiles

Antibiotic sensitivity profiles of the B. longum strain was determined using the ‘disc susceptibility’ assay. Cultures were grown up in the appropriate broth medium for 24-48 h spread-plated (1000 onto agar media and discs containing known concentrations of the antibiotics were placed onto the agar. Strains were examined for antibiotic sensitivity after 1-2 days incubation at 37° C. under anaerobic conditions. Strains were considered sensitive if zones of inhibition of 1 mm or greater were seen. The minimum inhibitory concentration (MIC) for each antibiotic was independently assessed. The MIC for clindamycin, vancomycin and metronidazole were 0.32, 0.75 and 0.38 respectively.

### Intestinal Transit

To determine whether Bifidobacterium longum could survive at low pH values equivalent to those found in the stomach, bacterial cells were harvested from fresh overnight cultures, washed twice in phosphate buffer (pH 6.5) and resuspended in TPY broth adjusted to pH 2.5 (with 1M HCl). Cells were incubated at 37° C. and survival measured at intervals of 5, 30, 60 and 120 minutes using the plate count method. AH1206 survived well for 5 minutes at pH 2.5 while no viable cells were recovered after 30 minutes.

Upon exiting the stomach, putative probiotics are exposed to bile salts in the small intestine. In order to determine the ability of B. longum to survive exposure to bile, cultures were streaked on TPY agar plates supplemented with 0.3% (w/v), 0.5%, 1%, 2%, 5%, 7.5% or 10% porcine bile. B. longum AH1206 growth was observed on plates containing up to 1% bile.

In a murine model, the ability of B. longum AH1206 to transit the gastrointestinal tract was assessed. Mice consumed 1×10⁹ AH1206 daily and faecal pellets were examined for the presence of the fed micro-organism. Detection of AH1206 was facilitated by isolating a spontaneous rifampicin resistant variant of the bifidobacteria-incorporation of rifampicin in the TPY plates used to assess transit ensured that only the fed rifampicin resistant bifiobacteria was cultured. Faecal samples were collected daily and B. longum transit through the gastrointestinal tract was confirmed (FIG. 2).

### Anti-Microbial Activity

The indicator pathogenic micro-organisms used in this study were propagated in the following medium under the following growth conditions: Salmonella typhimurium (37° C., aerobic) in Tryptone Soya broth/agar supplemented with 0.6% yeast extract (TSAYE, Oxoid), Campylobacter jejuni (37° C., anaerobic) and E. coli O157:H7 (37° C., anaerobic) on Blood agar medium, Clostridium difficile (37° C., anaerobic) in reinforced Clostridial medium (RCM, Oxoid). All strains were inoculated into fresh growth medium and grown overnight before being used in experiments.

Antimicrobial activity was detected using the deferred method (9). Briefly, B. longum AH1206 was incubated for 36-48 h. Ten-fold serial dilutions were spread-plated (100 μl) onto TPY agar medium. After overnight incubation, plates with distinct colonies were overlayed with the indicator bacterium. The indicator lawn was prepared by inoculating a molten overlay with 2% (v/v) of an overnight indicator culture which was poured over the surface of the inoculated TPY plates. The plates were re-incubated overnight under conditions suitable for growth of the indicator bacterium. Indicator cultures with inhibition zones greater than 1 mm in radius were considered sensitive to the test bacterium. B. longum AH1206 inhibited the growth of all pathogenic organisms tested, with zones of clearing measuring 14, >80, 13.33 and 17 mm for Salmonella typhimurium, Campylobacter jejuni, E. coli O157:H7 and Clostridium difficile respectively.

### Example 2

### Cytokine Production by PBMCs in Response to B. Longum

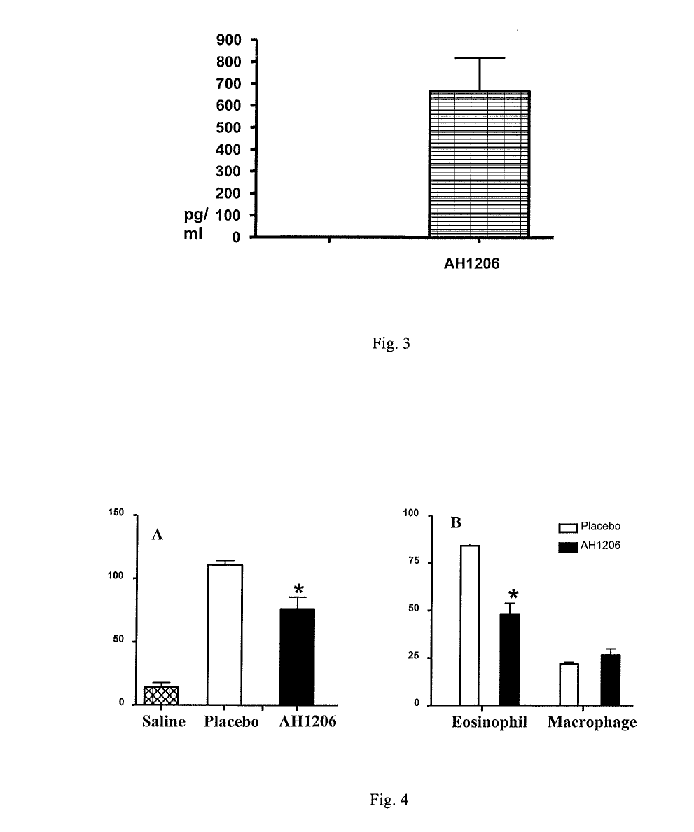

Peripheral blood mononuclear cells (PBMCs) were isolated from healthy donors by density gradient centrifugation. PBMCs were stimulated with the probiotic bacterial strain for a 72 hour period at 37° C. At this time culture supernatants were collected, centrifuged, aliquoted and stored at −70° C. until being assessed for IL-10 levels using cytometric bead arrays (BD BioSciences). AH1206 induced significant secretion of the anti-inflammatory cytokine IL-10 by human PBMCs (FIG. 3) suggesting this strain may be useful as a anti-inflammatory agent in vivo.

### Example 3

### B. longum AH1206 Attenuates Respiratory Disease in a Murine Model of Asthma

This study utilized a Balb/c ovalbumin (OVA) sensitized mouse model of allergic airway inflammation. Mice were sensitized by i.p. injection of OVA and disease was initiated by intranasal challenge with OVA. Twenty-four hours after the last challenge (day 15), mice were subjected to measurements of airway responsiveness followed by BAL procedure. OVA-sensitized, saline-challenged mice served as controls. Commencing on day 1 (i.e at time of first OVA sensitization), animals received B. longum AH1206 via a gavaging needle for 14 consecutive days. Animals gavaged with MRS broth served as controls.

Airway inflammation was assessed by inflammatory cell counts in bronchoalveolar lavage (BAL) fluid. Cells were removed from BAL fluid by centrifugation and cells were resuspended in phosphate-buffered saline (1 ml). BAL cells were stained with trypan blue, and viable cells were counted using a hemocytometer. Smears of BAL cells were prepared with a Cytospin (Thermo Shandon, Pittsburgh, Pa.) and stained with HEMA 3 reagent (Biochemical Sciences, Swedesboro, N.J.) for differential cell counts, where a total of 200 cells were counted for each lavage. Consumption of B. longum AH1206 significantly reduced the total BAL counts compared to placebo with the majority of this difference being seen in the eosinophil population (FIG. 4).

This study was repeated to further investigate whether the probiotic bacteria strain Bifidobacterium longum AH1206 suppresses allergic responses in an OVA sensitized mouse model of allergic airway inflammation. Briefly, adult male BALB/c mice were sensitized by i.p. injection of OVA day 0 and day 6. On days 12 and 14, mice were challenged intranasally with OVA. Twenty-four hours after the last challenge (day 15), mice were subjected to measurements of airway responsiveness followed by BAL procedure. OVA/alum-sensitized, saline-challenged mice served as controls. Animals received probiotic or placebo throughout the trial. Airway inflammation (cytokine and cell counts) was assessed by inflammatory cell counts in bronchoalveolar lavage (BAL) fluid. Airway responsiveness was also measured using the Buxco whole-body plethysmograph. Splenocytes were also isolated from OVA sensitized mice and were incubated in the presence of anti-CD3 and anti-CD28 antibodies after which cytokine levels were measured in the supernatants by flow cytometry.

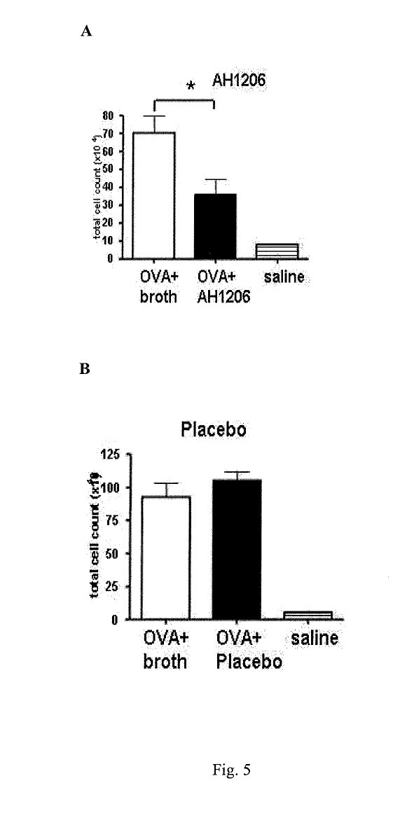

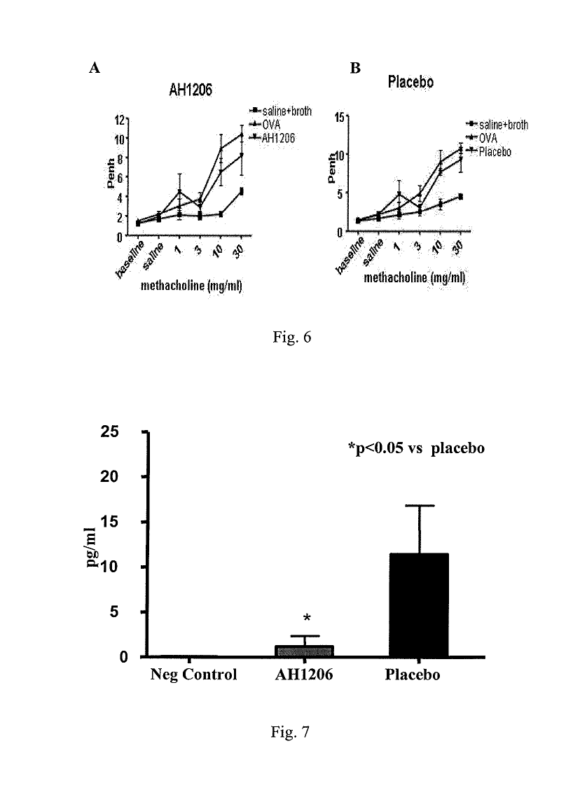

B. longum AH1206 treatment resulted in a significant reduction in cells recovered from BAL fluid following OVA challenge, when compared to broth fed animals (FIG. 5). Airway responsiveness was measured and challenge of sensitized mice with OVA resulted in an enhancement of AHR to methacholine when compared with saline-challenged mice. However no modulation of this enhanced airway responsiveness to methacholine, as assessed by changes in enhanced pause was seen (FIG. 6).

BAL cytokine levels were measured by cytometric bead array no significant differences were noted for IL-10, IFN-γ, IL-6 and CCL2 levels. AH1206 significantly reduced TNF-α levels compared to OVA control (FIG. 7).

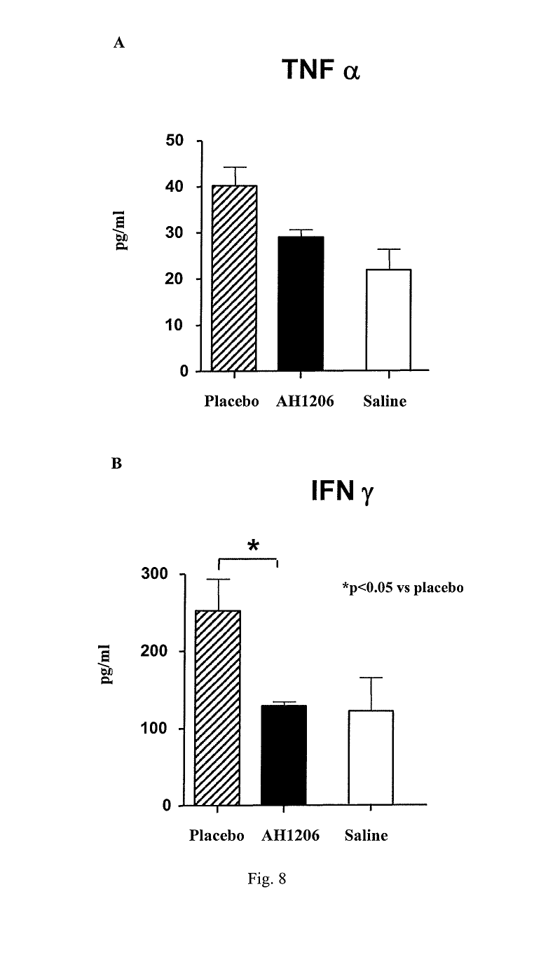

Cytokine levels in splenocyte supernatants were quantified by cytometric bead array following in vitro OVA or anti-CD3 anti-CD28 stimulation. Increased IL-10 release from OVA stimulated splenocytes, associated with in vivo OVA sensitization, was not observed in AH1206 fed mice. There was no significant difference in IL-6, TNF and MCP-1 (CCL2) levels. IL-10 release from CD3/CD28 splenocytes was not increased in AH1206 fed animals. However, secretion of the pro-inflammatory cytokines TNF-α and IFN-γ were significantly reduced in the splenocyte culture supernatants of AH1206-fed animals (FIG. 8). No significant changes were noted for the other cytokines measured.

### Example 4

### OVA Feeding Model

The aim of this study was to investigate whether the probiotic bacteria, Bifidobacterium longum AH1206 suppresses allergic responses in an ovalbumin (OVA)-induced allergy mouse model. BALB/c mice were divided into groups (8/group) and fed Placebo, Bifidobacterium longum AH1206 and Distilled H₂0 for four weeks. All mice were orally gavaged weekly with Ovalbumin and Cholera Toxin in 300 μls of PBS-excluding one of the dH₂0 groups which were orally gavaged with 300 μls PBS only as a control. After four weeks of treatment, a blood sample from each mouse was collected via facial vein puncture and a subsequent ELISA performed to measure OVA-specific IgE levels. The spleens and mesenteric lymph node cells were isolated and stimulated in vitro with LPS and antiCD3/CD28 and the immunodominant OVA peptide. Th1 and Th2 cytokines were measured by cytometric CBA.

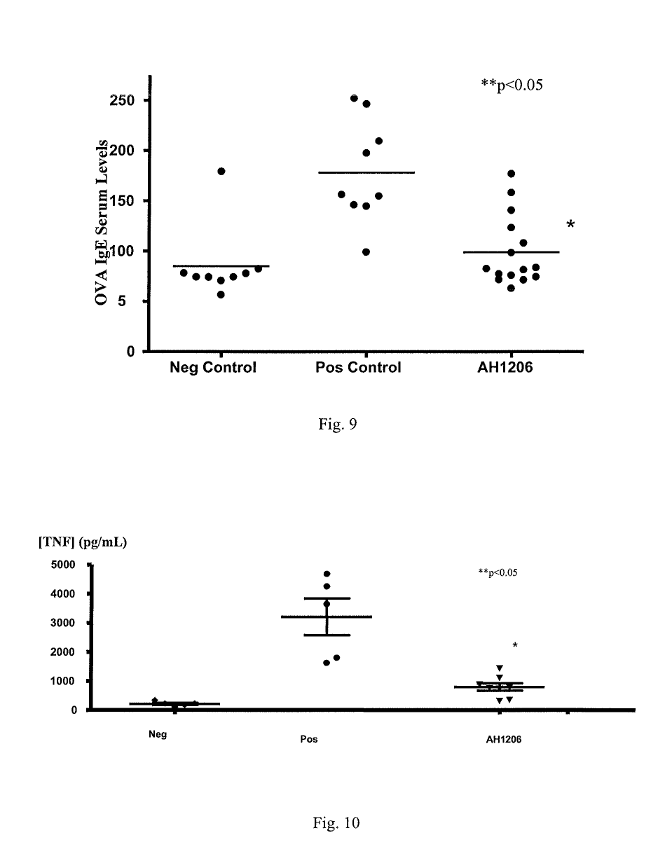

There was significantly less OVA-specific IgE induced in the probiotic fed group compared to the placebo and positive control groups (FIG. 9). The negative control group and the AH1206 fed groups were not different suggesting that AH1206 feeding completely inhibited the induction of an OVA-Specific IgE response. Statistics were done using the unpaired T test.

Splenocytes were isolated from probiotic, placebo and dH₂O fed BALB/c mice and either left unstimulated or stimulated with LPS, antiCD3/CD28 and the immunodominant OVA peptide and then analyzed for cytokine production of TNF-α, IL-2, IFN-γ, IL-4 and IL-5 by Th1/Th2 cytometric bead array. Cytokine results are summarized in Table 2.

TABLE 2
Cytokine summary

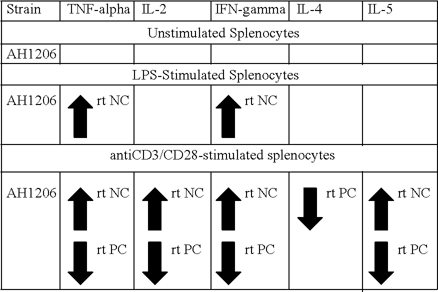

RT = Relative to
NC = Negative control (water fed, PBS challenged)
PC = Positive control (water fed, OVA and CT challenge

In un-stimulated splenocytes, no alterations were observed compared to control animals. TNF-α and IFN-γ release from LPS stimulated splenocytes was significantly greater for AH1206 fed animals compared to the negative controls but these levels were consistent with those observed with the OVA sensitized and cholera toxin administered positive controls. CD3/CD28 stimulation revealed profound alterations in lymphocyte signaling in the probiotic fed group. AH1206 fed animals secreted significantly less TNF-α compared to the positive controls but levels were higher compared to negative controls (FIG. 10). AH1206 fed animals had significantly lower levels of IFN-γ, IL-2, IL-4 and IL-5 compared to the non-probiotic fed positive controls.

### Example 5

### Treg Effector Model

This study investigated the effect of probiotic consumption on regulatory T cell number and activity in healthy mice. BALB/c mice (10/group) were fed Bifidobacterium longum AH1206 or placebo for three weeks. Following probiotic/placebo consumption, CD4+CD25+ T-regulatory cells were isolated and their in vitro suppressive activity was determined by measuring proliferation of anti-CD3/CD28 stimulated CFSE-labelled CD4+ responder T cells using flow cytometry. CD4+ responder T cells were co-incubated with CD4+CD25− T cells as a control. The percentage of CD4+CD25+ cells (Regulatory T cells) in murine splenocytes that are also FoxP3 positive was determined in the spleens of probiotic or placebo-fed mice.

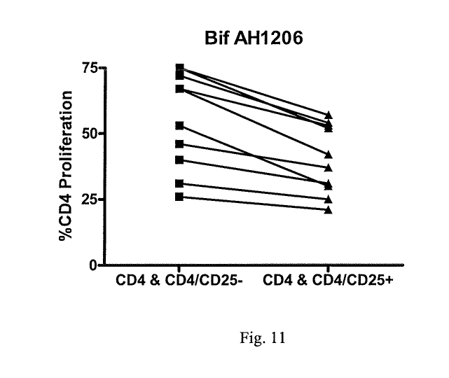

The % of CD4+ cells that proliferated when co-incubated with CD4+CD25+ cells from the probiotic/placebo fed mice was compared to the % of CD4+ cells that proliferated when co-incubated with CD4+CD25− cells from the same trial mouse. In each case, T cell proliferation was less in cultures containing CD4+CD25+ cells compared in cultures containing CD4 cells alone and depleted of the CD25+ cells (FIG. 11).

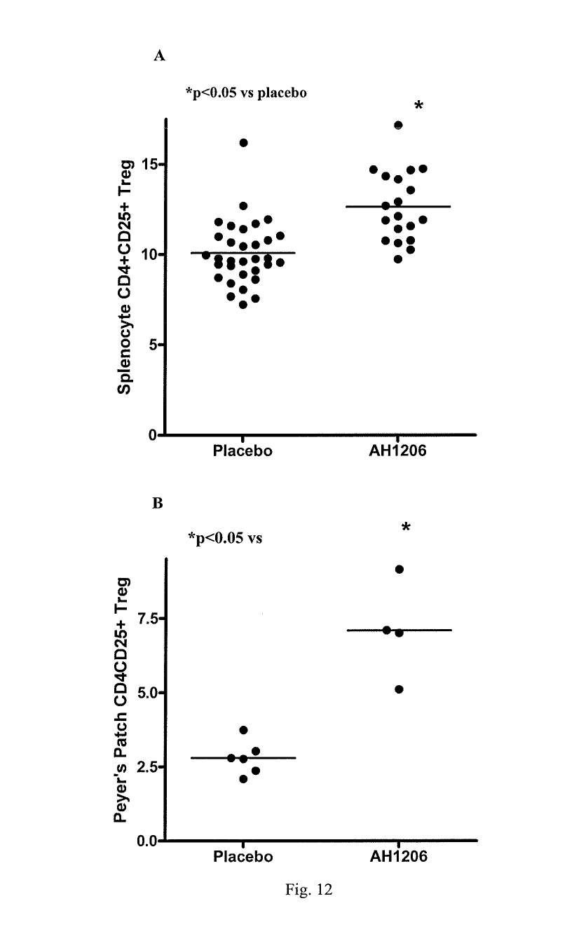

The % of cells in the CD4+ population that were also CD25+ was determined (FIG. 12). The Bifidobacterium longum AH1206 fed group had significantly more CD4+ T cells that were CD25+ (i.e. T-Regulatory cells) than their placebo-fed counterparts (p=0.0081). This suggests that the % of T-Regulatory cells within the CD4+ population was increased significantly by feeding with AH1206.

The number of CD4+CD25+FoxP3+ cells in the whole splenocyte populations of probiotic or placebo-fed mice was also determined. The number of CD4+CD25+ T-Regulatory cells expressing FoxP3 was unchanged in the spleens of probiotic fed mice relative to placebo or unfed mice

### Example 6

### Germ Free Model

Germ free mice were purchased at 6 weeks of age and maintained in the germ-free unit at the biological services unit in UCC. Animals consumed the probiotic strain Bifidobacterium longum AH1206 for 14 days or remained germ free. Induction of T regulatory cells was assessed by flow cytometry.

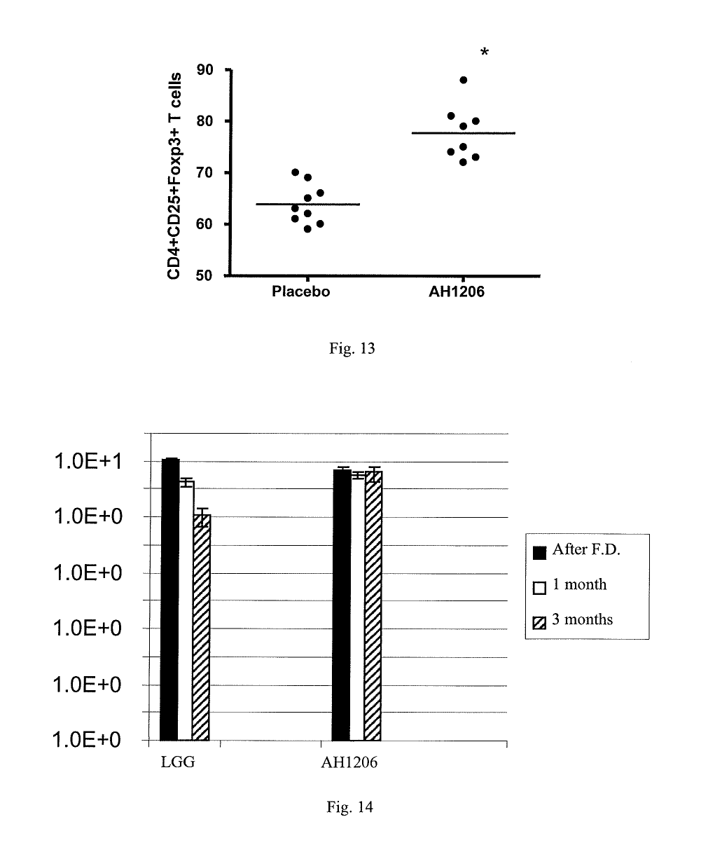

The numbers of CD4+CD25+Foxp3+ cells in the spleen of AH1206 fed germ-free animals was significantly increased following feeding (FIG. 13). Total CD3/CD4 or CD3/CD8 counts remained unaltered.

### Example 7

### Stability Results

The stability of probiotic strain-AH1206 was assessed over 3 months at 30° C. (FIG. 13).

These results indicate that Lactobacillus rahmosus GG was a poor performer over the test period with a 2 log drop over the 3 month period whereas AH1206 was quite stable with no viability loss recorded over the period.

### Example 8

### Murine Allergy Models

Atopic allergic sensitization is characterised by production of IgE against environmental antigens such as house dust mites, grass pollen, and animal proteins and can lead to a range of allergic disorders including asthma, rhinitis and atopic dermatitis [20]. These disorders affect 10-15% of Western populations, and their prevalence has doubled in the last 10-15 years. While many hypotheses have been proposed to explain this phenomenon, a significant body of evidence now supports the hygiene hypothesis-originally proposed by Strachan in 1989 [21]. The original hypothesis proposed that allergic diseases were prevented by early infectious exposure introduced by contact with older siblings. The mechanism was thought to include the potential effect of pathogenic microbes on polarising the type 2 T helper (Th2) cell biased cytokine response of the human neonate towards a type 1 T helper (Th1) cell response. More recently, the hygiene hypothesis has expanded beyond the involvement of pathogenic microbes to include the gastrointestinal microbiota which are important participants in normal immunological development and function. Understanding of the mechanism has also evolved, with reduced T regulatory cell activity now considered equally important in the immune dysregulation associated with allergy [22, 23]. Because of this, there is increased interest in elucidating the role of specific organisms in modulating host immune processes and ultimately, their impact on allergic manifestations.

Select representatives of the gastrointestinal microbiota have been shown to exert immunoregulatory activity both in animal models and in human studies [24-27]. Human clinical trials demonstrated that Lactobacillus rhamnosus GG could prevent early atopic disease [28, 29] and Lactobacillus fermentum was shown to improve the extent and severity of atopic dermatitis in young children [30]. In contrast, Lactobacillus rhamnosus GG was ineffective when administered to young adults with birch-pollen allergy, suggesting that administration early in life may provide the optimal beneficial effect [30].

T regulatory cells are derived from the thymus but may also be induced in peripheral organs, including the gut mucosa [31, 32]. CD103⁺ dendritic cells within the mucosa are largely responsible for the conversion of T regulatory cells via TGF-β and retinoic acid dependent processes [33, 34]. The conversion is likely driven by gastrointestinal specific environmental factors associated with the presence of large numbers of commensal organisms. For example, encounters with specific experimental microbes within the murine gut, have been shown to drive the development of mucosal T regulatory cells which is associated with attenuation of inflammation in a murine model of colitis [35]. In addition, the consumption of a Bifidobacterium infantis strain promotes T regulatory cell conversion and protects against LPS-induced NF-κB activation in vivo while Lactobacillus reuteri induces T regulatory cells which protect against an allergic airway response in mice [36, 37]. However, it is unlikely that all commensal microbes are equally effective in inducing T regulatory cells in vivo.

The purpose of this study was to use murine models to examine the impact of consuming three different microbes—Bifidobacterium breve AH1205, Bifidobacterium longum AH1206 and Lactobacillus salivarius AH102—on immunological and allergy outcomes including: (a) the induction of T regulatory cells; (b) the relative effectiveness of consumption during the perinatal period, in adulthood or under germ-free conditions; and (c) the development of allergy and its correlation with T regulatory cell induction.

### Methods

### Bacterial Strains

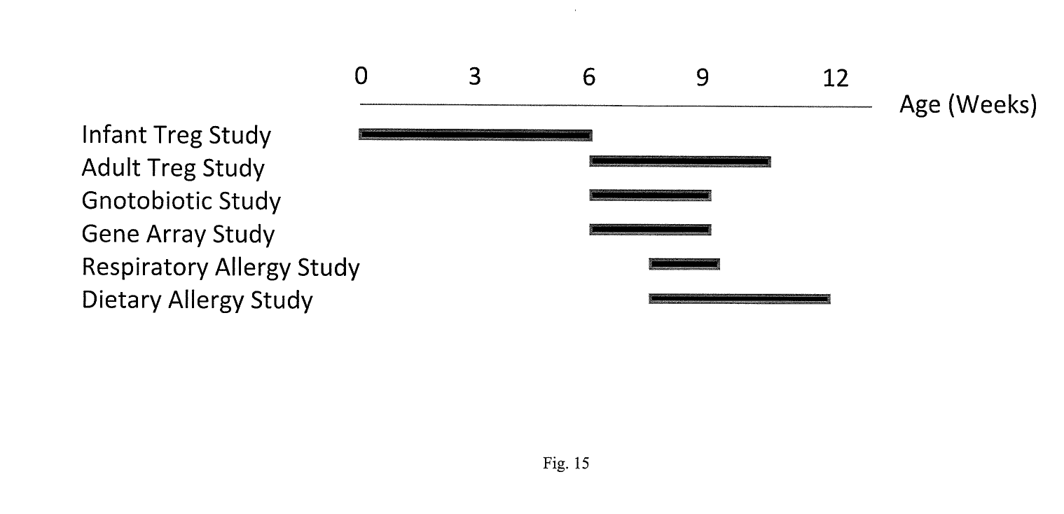

Bifidobacterium breve AH1205 (Bifidobacterium AH1205), Bifidobacterium longum AH1206 (Bifidobacterium AH1206) and Lactobacillus salivarius AH102 (Lactobacillus AH102) were obtained from the Alimentary Health Ltd (Cork, Ireland) culture collection. All strains are of human origin and were originally isolated from infant faecal samples (Bifidobacterium AH1205 and AH1206) or healthy adult resected intestinal tissue (Lactobacillus AH102). Bacteria were routinely cultured anaerobically for 48 hours in deMann, Rogosa and Sharpe medium, MRS, (Oxoid, Basingstoke, UK). Bifidobacteria strains were supplemented with 0.05% cysteine (Sigma, Dublin, Ireland). Freeze-dried powders were generated for each strain by resuspending the centrifuged pellet in a cryoprotectant solution (18% reconstituted skim milk, 2% sucrose), followed by freezing at −20° C. for 24 h and lyophilisation for another 24 h. Lyophilised bacteria were resuspended in sterile water and were administered orally, via a pipette tip, to the mice at a final dose of approximately 2×10⁹ colony forming units per day. Spontaneous rifampicin resistant variants were administered in representative experiments in order to enumerate faecal levels on selective rifampicin MRS plates as previously described [38]. All animal experiments were approved by the University College Cork ethics committee and were conducted under appropriate licence from the Irish government. An overview of the different animal models used is illustrated in FIG. 15.

### Assessment of T Regulatory Cell Induction in Healthy Mice

Bifidobacterium AH1205, Bifidobacterium AH1206 and Lactobacillus AH102 were administered daily to BALB/c mice immediately following birth (within 48 hours) for a period of 6 weeks. In addition, adult (6 week old) BALB/c mice were administered the commensal strains daily for 4 weeks. Adult Swiss Webster germ-free animals were administered an individual bacterial strain (thereafter termed gnotobiotic animals—defined as an animal in which only certain known strains of micro-organisms are present) for 3 weeks and examined for T regulatory cell induction within the spleen.

At the end of each feeding period, Peyer's patches and/or spleens were isolated and single cell suspensions generated by mechanical means. Monoclonal antibodies to CD3, CD4, and CD25 (BD Biosciences, Oxford, UK) were used to label cells for T cell subset analysis. Antibodies to the transcription factor Foxp3 (eBioscience, San Diego, USA) were used to label permeabilised cells. Cellular phenotypes were measured using a BD FacsCaliber flow cytometer and analysis was performed using BD CellQuest software.

The suppressive activity of CD4/CD25+ T cells from the adult microbe-fed animals was determined by assessing their inhibitory effect on T cell proliferation. Naïve CD4 T cells were stimulated with anti-CD3 and anti-CD28 antibodies (BD) and co-incubated with CD4+CD25+ T cells from Bifidobacterium AH1206-fed animals for three days. CD4 T cell proliferation was measured using the CellTrace™ CFSE Cell Proliferation Kit (Invitrogen, Carlsbad, USA). Lymphocyte proliferation was measured by flow cytometry.

### Gene Array Analysis of Bifidobacterium AH1206-Fed Animals

In order to investigate the immunological basis for Bifidobacterium AH1206 induction of T regulatory cells, Peyer's patches were obtained from adult Bifidobacterium AH1206-fed animals (n=5) and placebo-fed animals (n=5) following 3 weeks treatment. Total RNA was isolated from tissue using the Absolutely RNA RT-PCR Miniprep kit (Stratagene, La Jolla, USA) and RNA quantity and quality was confirmed by microspectrophotometry (Nanodrop, Wilmington, USA) and electrophoresis (Agilent 2100 Bioanalyser, Agilent Technologies, Santa Clara, USA) respectively. Biotin labeled cRNA was hybridized to GeneChip Human Genome U133 Plus 2.0 Arrays (Affymetrix, High Wycombe, UK) in accordance with the manufacturer's instructions. Following washing and staining steps with streptavidin-c (Molecular Probes, Eugene, USA), probe arrays were scanned using the GeneChip system Affymetrix confocal scanner. Significance analysis was performed using Array Assist software (Stratagene) to determine expression changes between feeding groups. Differential gene expression was determined based on Bifidobacterium AH1206 versus placebo treatment gene expression change (two-fold minimum cut-off and p<0.05).

### OVA Respiratory Allergy Model

Adult BALB/c mice (20-25 g) were sensitized by i.p. injection of 20 μg OVA absorbed with 500 μg alum in saline on day 0 and day 6. On days 12 and 14, mice were challenged intra-nasally with 5 μg OVA per mouse. Twenty-four hours after the last challenge (day 15), bronchoalveolar lavage (BAL) was obtained from all animals. Mice, OVA/alum-sensitized, but saline-challenged intra-nasally, served as negative controls, while bacterial-free, MRS broth-fed animals served as placebo controls. Animals received approximately 2×10⁹ CFU/day Bifidobacterium AH1205, Bifidobacterium AH1206 or Lactobacillus AH102 via a gavaging needle for 14 consecutive days. Airway inflammation was assessed by BAL inflammatory cell counts. Cells were removed from BAL fluid by centrifugation and supernatants were stored at −80° C. until evaluation of cytokine content. BAL cells were prepared with a Cytospin (Thermo Shandon, Pittsburgh, USA) and stained with HEMA 3 reagent (Biochemical Sciences, Swedesboro, USA) for differential cell counts, where a total of 200 cells were counted for each lavage. BAL cytokine levels (IL-6, IL-10, IL-12, MCP-1, TNF, IFNγ) were assessed using the BD™ cytometric bead array system (BD biosciences, San Jose, USA).

### OVA Dietary Allergy Model

BALB/c mice (8 weeks old) were gavaged on days 7, 14 and 21 with 5 mgs OVA and 10 μgs Cholera Toxin (in order to overcome oral tolerance) while the negative controls were gavaged with PBS only. Bifidobacterium AH1205, Bifidobacterium AH1206 and Lactobacillus AH102 were administered daily during the 4-week study period. Serum was obtained from each mouse via facial vein puncture and OVA-specific IgE levels determined by ELISA according to manufacturer's instructions (MD-Biosciences, Zurich, Switzerland). In addition, splenocytes were isolated and stimulated in vitro with anti-CD3 and anti-CD28 antibodies (BD). Supernatant cytokine levels were quantified by CBA as described above.

### Statistics

GraphPad Prism software utilising 2Way-ANOVA with Bonferroni's Post-test was used to determine statistical significance.

### Results

### Microbial Induction of T Regulatory Cells

The three organisms, Bifidobacterium AH1205, Bifidobacterium AH1206 and Lactobacillus AH102, were fed to infant mice, adult mice and germ-free mice in order to comprehensively assess their capacity to induce T regulatory cells under different experimental conditions.

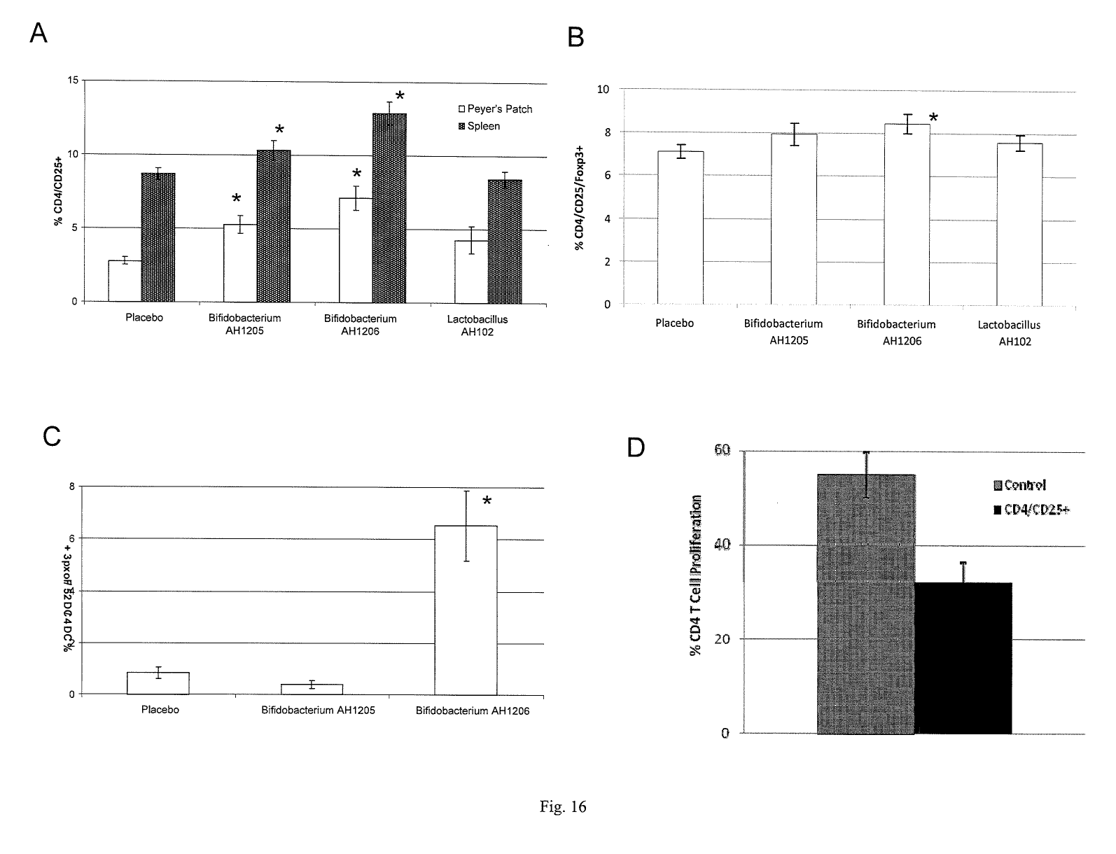

Consumption of Bifidobacterium AH1205 and Bifidobacterium AH1206 significantly increased the percentage of Peyer's patch CD4/CD25+ T cells (p=0.003 and p=0.0003 respectively) and splenic CD4/CD25+ T cells (p=0.04 and p=0.0001 respectively) when administered from birth (FIG. 16a). Representative samples were also stained for Foxp-3 expression revealing that 66%, 71%, 68% and 69% of splenic CD4/CD25+ cells were also Foxp3+ in the placebo, Bifidobacterium AH1205, Bifidobacterium AH1206 and Lactobacillus AH102 groups respectively. Associated with the increase in CD4/CD25+ T cells, splenocytes released greater amounts of IL-10 when stimulated with antiCD3/CD28 in vitro compared to control placebo-fed animals (Table 3). However, only Bifidobacterium AH1206 retained the ability to increase splenic CD4/CD25/Foxp3+ T cells by when consumed by adult mice (placebo 7.1+/−0.3% versus AH1206 8.5+/−0.4%; p=0.02, FIG. 16b). This represents a 19% increase in the proportion of splenic CD4/CD25/Foxp3+ T cells in Bifidobacterium AH1206-fed animals compared to placebo. Lactobacillus AH102 did not influence splenic T cell populations under any of the conditions tested. Mono-colonisation of gnotobiotic animals with Bifidobacterium AH1206 (p=0.0001), but not Bifidobacterium AH1205, significantly increased the numbers of splenic CD4/CD25/Foxp3+ T cells (FIG. 16c).

In order to confirm that the CD4/CD25+ cells did indeed exert a regulatory function, these cells were tested for their ability to suppress T cell proliferation. Isolated CD4/CD25+ T cells from Bifidobacterium AH1206-fed adult animals effectively suppressed T cell proliferation (FIG. 16d).

Gastrointestinal Survival does not Correlate with T Regulatory Cell Induction

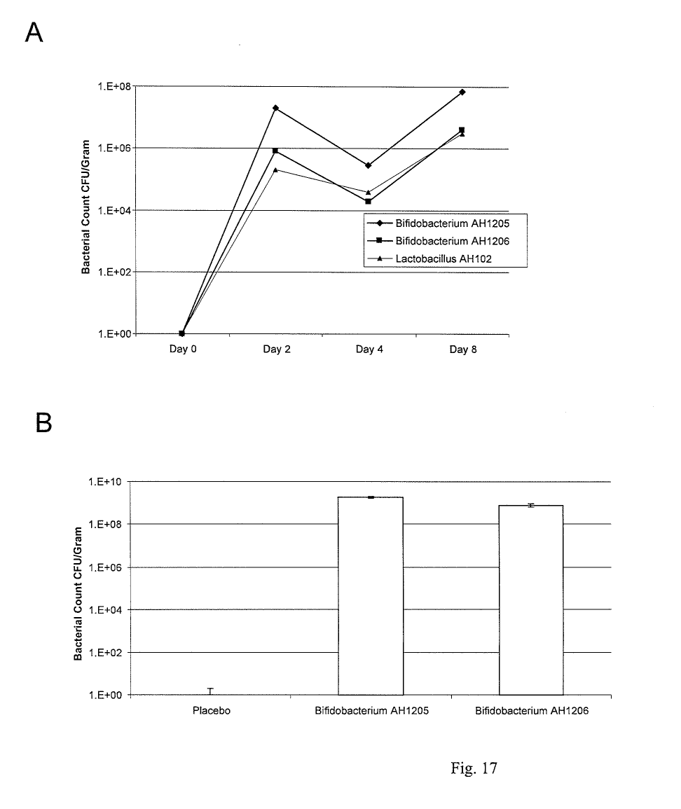

One potential explanation for the selective enhancement of T regulatory cells by a microbial strain is that greater numbers of that bacterial strain survives within the gut and therefore have a greater impact on host immune signalling. Gastrointestinal transit, as determined by culturing faecal samples for rifampicin resistant colonies, was similar for Bifidobacterium AH1205, Bifidobacterium AH1206 and Lactobacillus AH102 in adult mice that consumed these bacterial strains for one week (FIG. 17a). In addition, faecal recovery of Bifidobacterium AH1205 and Bifidobacterium AH1206 was identical from germ-free mice (FIG. 17b).

Gene array analysis of Bifidobacterium AH1206-fed animals Following consumption of Bifidobacterium AH1206, 53 genes were differentially expressed in Peyer's patches. Genes were organised according to biological function and are illustrated in Table 4. Differential expression was noted for nine genes related to immunological processes (8 decreased and 1 increased expression), 27 genes associated with metabolism (6 decreased and 21 increased expression), 5 genes associated with DNA transcription (2 decreased and 3 increased expression), 3 genes related to apoptosis (all decreased) and 1 gene involved in signal transduction (decreased expression). The biological functions of 8 differentially expressed genes are not known and are not presented. The immunological related genes that were suppressed included CEACAM1 (expressed on activated T cells [39]), GPx2 (a glutathione peroxidase involved in the adaptive response [40]), H2-T18 (involved in MHC class I receptor activity), NF-κBIζ (positive regulator of TLR/IL-1R-mediated IL-6 production through association with NF-κB p50 [41]), Osp94 (osmotic stress response gene [42]), REGIIIγ (C-type lectin with anti-microbial activity [43]), SOCS3 (regulator of cytokine signalling [44]) and T2BP (activates NF-κB following TNF-α stimulation [45]). In contrast, increased expression of the nuclear hormone receptor PPARα may play a role in the anti-inflammatory effect associated with this bacterium [46]). In addition, increased expression of genes involved in retinoic acid metabolism (Aldh1a1, Aldh1a3 and Rdh7) within the Peyer's patch suggest that nuclear hormone receptor signalling is significantly modulated by Bifidobacterium AH1206 consumption.

### Bifidobacterium AH1206 Reduces Inflammatory Cell Recruitment in the OVA Respiratory Allergy Model

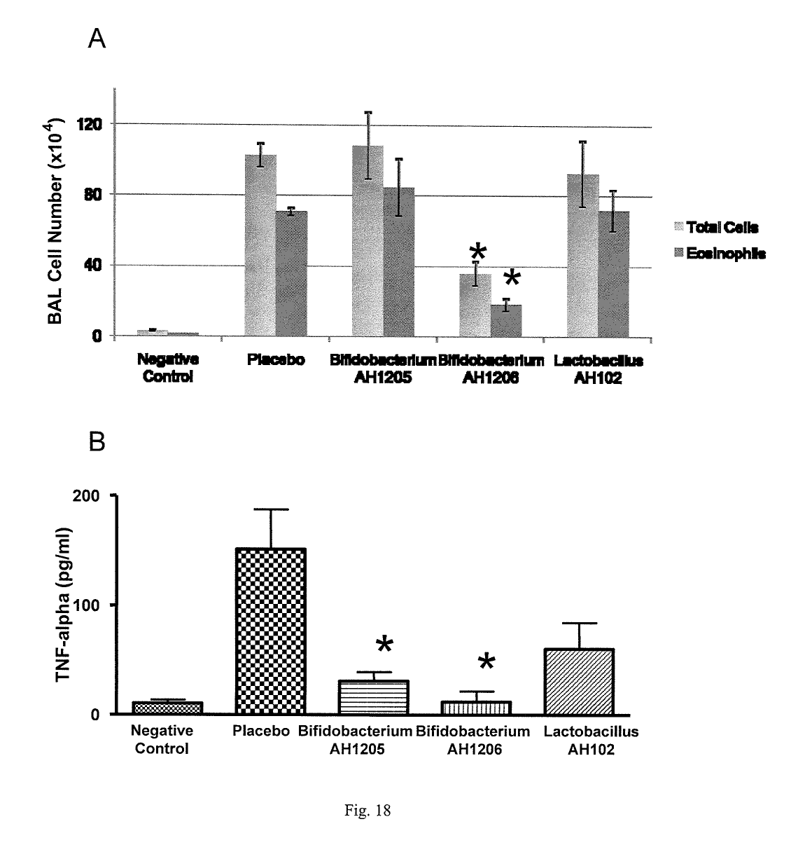

Consumption of Bifidobacterium AH1206, but not Bifidobacterium AH1205 or Lactobacillus AH102, significantly reduced BAL inflammatory cell numbers following OVA challenge (FIG. 18a). Differential cell counts revealed that the reduction in inflammatory cells was primarily due to a reduction in eosinophil recruitment (FIG. 18a). Assessment of cytokine levels in BAL revealed a significant reduction in TNF-α levels for both Bifidobacterium AH1205 (31+/−8 μg/ml) and Bifidobacterium AH1206 (11+/−9 pg/ml) with no change observed with Lactobacillus AH102 consumption (60+/−24 pg/ml) compared to the placebo group (151+/−36 μg/ml, FIG. 18b). BAL IL-6 levels were also significantly reduced in the Bifidobacterium AH1206 group (8.7+/−8 μg/ml compared to the placebo group 44.5+/−15 μg/ml). No differences were observed for IFN-γ (1.8+/−0.9 versus 1.1+/−0.9 μg/ml), CCL-2 (25+/−21 versus 23+/−5 μg/ml) or IL-10 (4+/−4 versus 5+/−3 μg/ml) levels in BAL from placebo or Bifidobacterium AH1206-fed animals respectively.

### Bifidobacterium AH1206 Suppresses the IgE Response to OVA

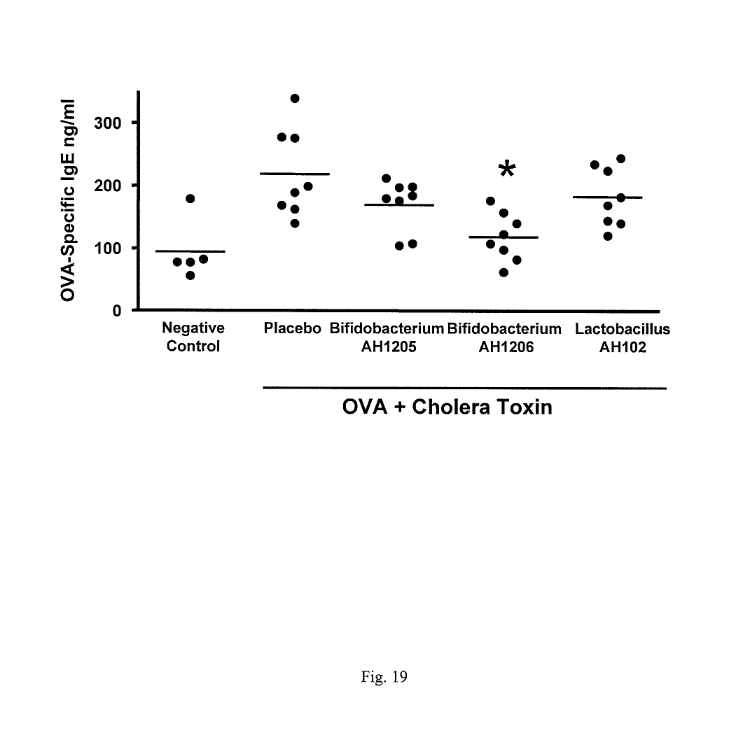

Using a model of dietary allergy whereby OVA and cholera toxin are administered orally, we determined the influence of microbial feeding on the serum IgE response to OVA. Serum OVA-specific IgE levels were significantly increased following OVA-CT administration (FIG. 19). Consumption of Bifidobacterium AH1206 suppressed the increase in IgE levels to OVA while Bifidobacterium AH1205 and Lactobacillus AH102 had no effect (FIG. 19).

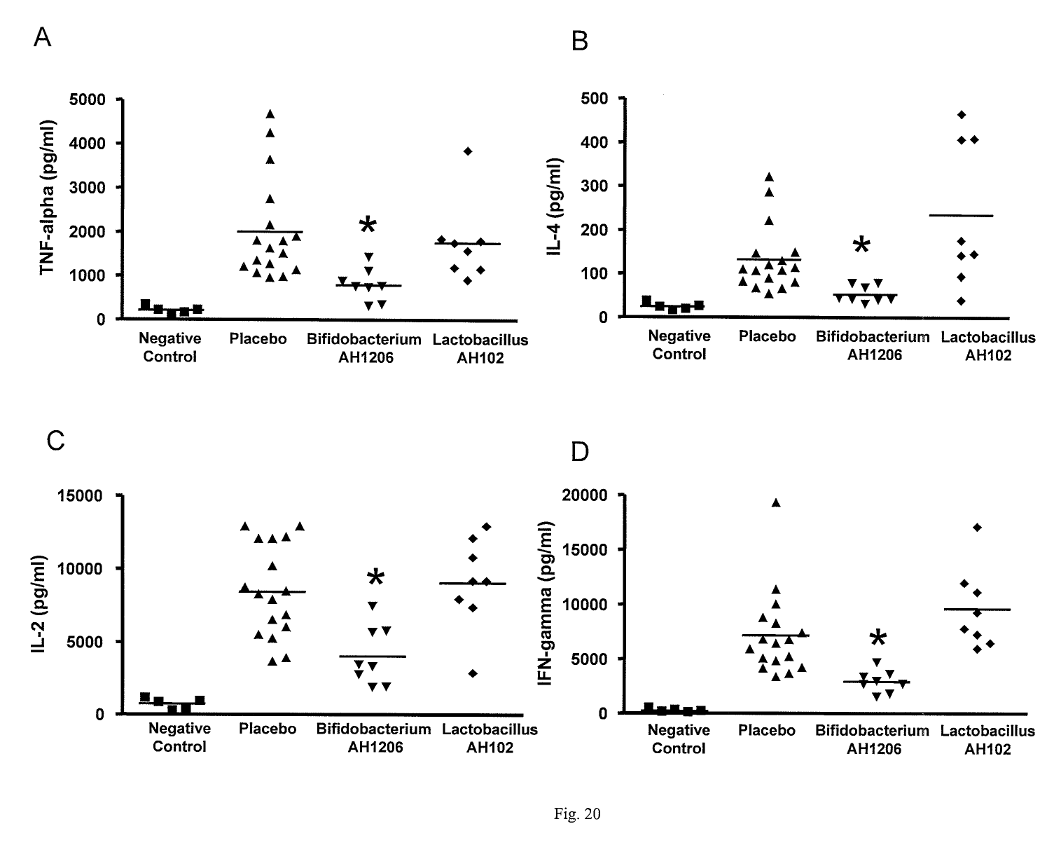

Splenocytes were isolated from OVA-CT sensitised animals and stimulated in vitro with anti-CD3 and anti-CD28 antibodies. Splenocyte secretion of TNF-α, IL-2, IL-4 and IFN-γ was significantly reduced for splenocytes isolated from Bifidobacterium AH1206-fed animals compared to placebo-fed controls (FIG. 20). Lactobacillus AH102 consumption had no effect on splenocyte cytokine secretion in this model.

### Discussion

In this study we compared three different organisms, Bifidobacterium breve AH1205, Bifidobacterium longum AH1206 and Lactobacillus salivarius AH102, for their ability to induce T regulatory cells and their impact on respiratory and oral allergy development in murine models. Bifidobacterium AH1206 augmented T regulatory cell numbers when administered perinatally, to adult mice and in a gnotobiotic setting. Bifidobacterium AH1205 enhanced Peyer's Patch and splenic T regulatory numbers only when administered from birth, while Lactobacillus AH102 was ineffective at increasing T regulatory cell numbers under all conditions examined. In addition, consumption of Bifidobacterium AH1206 protected against eosinophil recruitment to the lung and blocked induction of serum IgE. Neither Bifidobacterium AH1205 nor Lactobacillus AH102 protected against allergic inflammatory activity.

The induction of T regulatory cells by Bifidobacterium AH1205 was only observed when this bacterium was fed to mice in the perinatal period and not when mice reached adulthood. In addition, induction of T regulatory cells by Bifidobacterium AH1206 was more pronounced when consumed in the perinatal period. This is an interesting observation and suggests that age-dependent host-specific processes such as immunological maturity, mucosal lymphoid antigen sampling and/or gut barrier integrity may be important for the microbial induction of host regulatory responses. It is unlikely that competition with an already established microbiota is solely responsible for the reduced effect in adult mice as mono-colonisation of adult gnotobiotic mice with Bifidobacterium AH1205 did not result in an increase in T regulatory cell numbers. Interestingly, faecal transit in gnotobiotic animals was approximately two logs higher for both Bifidobacteria, compared to the levels observed in conventionally colonised animals.

In order to further characterise the biological basis for Bifidobacterium AH1206 induction of T regulatory cells, we performed gene array analysis of the primary antigen sampling site within the gut, the Peyer's patches. Consistent with the observation of increased T regulatory cells in these animals, immune related gene expression changes were largely suppressed and included genes associated with antigen presentation, TLR activation and cytokine signalling. In addition, genes associated with the metabolism of retinoic acid were increased and it is tempting to speculate that dendritic cell secretion of retinoic acid may contribute to the observed increase in T regulatory cell numbers following Bifidobacterium AH1206 consumption as retinoic acid stabilises Foxp3 expression [33, 34].

The appropriate balance between Th1 and Th2 cytokines is an important factor influencing the development of allergy. However, Bifidobacterium AH1206 suppressed secretion of TNF-α, IL-2, IL-4 and IFN-γ from stimulated splenocytes suggesting that a selective increase in Th1 cytokines was not responsible for the protective effect in the allergy models. In contrast, other microbes that inhibit the IgE response to OVA exert their effect via up-regulation of the Th1 response [47]. Our data suggests that the mechanism underpinning the Bifidobacterium AH1206 protective effect is likely to include T regulatory cells which would suppress aberrant activation of both Th1 and Th2 responses.

Multiple studies in animal models indicate that T regulatory cells play an important role in regulating allergen-specific inflammatory responses. CD4+CD25+Foxp3+ cells are recruited into both lungs and draining lymph nodes and can suppress allergen induced airway eosinophillia, mucous hypersecretion and airway hyper-responsiveness [48-53]. In addition, the natural resolution of an allergic airway response to Der p1 in mice was shown to be dependent on CD4+CD25+Foxp3+ cells that appear in lungs and draining mediastinal lymph nodes following airway challenge. Successful immunotherapy in humans is also associated with the induction of peripheral allergen tolerance involving IL-10 and TGF-β secreting T regulatory cells [54]. In addition, high dose allergen exposure in healthy humans is associated with clonal expansion of T regulatory cells which may switch in vivo from existing Th1- and Th2-like allergen specific cells [55].

Bifidobacteria and lactobacilli are among the early and important colonizers of the gastrointestinal tract and are generally considered to be part of a normal, healthy microbiota. Our results provide further support for the concept that interactions between specific non-pathogenic micro-organisms and the host can lead to major immune modulating events, the consequences of which are not confined to the gastrointestinal tract. The induction of protective regulatory responses was shown to be influenced by the bacterial strain itself, the age of the host and possibly by the existing microbiota that are already present within the gastrointestinal tract.

While the mechanisms responsible for microbiota associated regulation of aberrant immune-reactivity are not well understood, it has been proposed that effective immunotherapeutic microbes may exert their influence via modulation of T regulatory cells. Bifidobacterium AH1206 was shown to mediate potent activation of the T regulatory cell program. While further examination of the mechanisms underpinning this phenomena are required to identify the specific ligand-receptor interactions that mediate this activity, clinical studies in appropriate populations appear warranted to evaluate it's significant potential in the prevention and treatment of allergy in humans.

TABLE 3
Splenocyte secretion of IL-10

| Treatment | IL-10 (pg/ml) |
|---|---|
| Placebo | 129 +/− 21 |
| Bifidobacterium AH1205 | 314 +/− 55* |
| Bifidobacterium AH1206 | 244 +/− 34* |
| Lactobacillus AH102 | 150 +/− 22 |

Splenocytes were stimulated in vitro with anti-CD3 and anti-CD28 antibodies and supernatant IL-10 levels quantified. Consumption of Bifidobacterium AH1205 and Bifidobacterium AH1206 was associated with an increased in vitro secretion of IL-10.
Results are expressed as the mean +/− SE per group (n = 10 per group)
*p < 0.05 compared to placebo

Splenocytes were stimulated in vitro with anti-CD3 and anti-CD28 antibodies and supernatant IL-10 levels quantified. Consumption of Bifidobacterium AH1205 and Bifidobacterium AH1206 was associated with an increased in vitro secretion of IL-10. Results are expressed as the mean+/−SE per group (n=10 per group) *p<0.05 compared to placebo

TABLE 4
Peyer's Patch Gene Expression Patterns

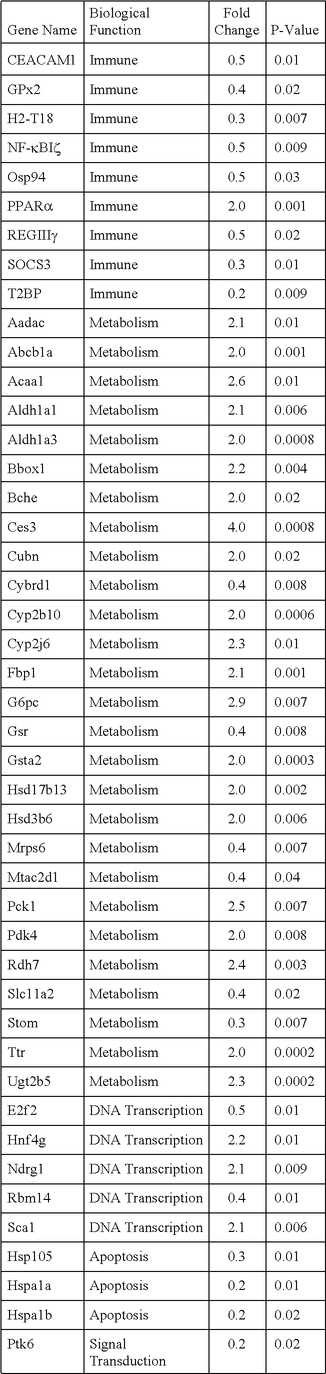

Following consumption of Bifidobacterium AH1206, changes in gene expression were identified using gene arrays. Gene changes are grouped according to biological function and genes with increased expression are highlighted.

Results are expressed as the mean fold change in gene expression compared to placebo (n=5 placebo and n=5 Bifidobacterium AH1206-fed animals).

### Immunomodulation

The human immune system plays a significant role in the aetiology and pathology of a vast range of human diseases. Hyper and hypo-immune responsiveness results in, or is a component of, the majority of disease states. One family of biological entities, termed cytokines, are particularly important to the control of immune processes. Pertubances of these delicate cytokine networks are being increasingly associated with many diseases. These diseases include but are not limited to inflammatory disorders, immunodeficiency, inflammatory bowel disease, irritable bowel syndrome, cancer (particularly those of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and acne vulgaris. The effects on cytokine production are specific for the probiotic strain-examined. Thus specific probiotic strains may be selected for normalising an exclusive cytokine imbalance particular for a specific disease type.

Customisation of disease specific therapies can be accomplished using either a single strain of AH1206 or mutants or variants thereof or a selection of these strains.

### Immune Education

The enteric flora is important to the development and proper function of the intestinal immune system. In the absence of an enteric flora, the intestinal immune system is underdeveloped, as demonstrated in germ free animal models, and certain functional parameters are diminished, such as macrophage phagocytic ability and immunoglobulin production (10). The importance of the gut flora in stimulating non-damaging immune responses is becoming more evident. The increase in incidence and severity of allergies in the western world has been linked with an increase in hygiene and sanitation, concomitant with a decrease in the number and range of infectious challenges encountered by the host. This lack of immune stimulation may allow the host to react to non-pathogenic, but antigenic, agents resulting in allergy or autoimmunity. Deliberate consumption of a series of non-pathogenic immunomodulatory bacteria would provide the host with the necessary and appropriate educational stimuli for proper development and control of immune function.

### Inflammation

Inflammation is the term used to describe the local accumulation of fluid, plasma proteins and white blood cells at a site that has sustained physical damage, infection or where there is an ongoing immune response. Control of the inflammatory response is exerted on a number of levels (11). The controlling factors include cytokines, hormones (e.g. hydrocortisone), prostaglandins, reactive intermediates and leukotrienes. Cytokines are low molecular weight biologically active proteins that are involved in the generation and control of immunological and inflammatory responses, while also regulating development, tissue repair and haematopoiesis. They provide a means of communication between leukocytes themselves and also with other cell types. Most cytokines are pleiotrophic and express multiple biologically overlapping activities. Cytokine cascades and networks control the inflammatory response rather than the action of a particular cytokine on a particular cell type (12). Waning of the inflammatory response results in lower concentrations of the appropriate activating signals and other inflammatory mediators leading to the cessation of the inflammatory response. TNFα is a pivotal proinflammatory cytokine as it initiates a cascade of cytokines and biological effects resulting in the inflammatory state. Therefore, agents which inhibit TNFα are currently being used for the treatment of inflammatory diseases, e.g. infliximab.

Pro-inflammatory cytokines are thought to play a major role in the pathogenesis of many inflammatory diseases, including inflammatory bowel disease (IBD). Current therapies for treating IBD are aimed at reducing the levels of these pro-inflammatory cytokines, including IL-8 and TNFα. Such therapies may also play a significant role in the treatment of systemic inflammatory diseases such as rheumatoid arthritis.

The strains of the present invention may have potential application in the treatment of a range of inflammatory diseases, particularly if used in combination with other anti-inflammatory therapies, such as non-steroid anti-inflammatory drugs (NSAIDs) or Infliximab.

### Cytokines and Cancer

The production of multifunctional cytokines across a wide spectrum of tumour types suggests that significant inflammatory responses are ongoing in patients with cancer. It is currently unclear what protective effect this response has against the growth and development of tumour cells in vivo. However, these inflammatory responses could adversely affect the tumour-bearing host. Complex cytokine interactions are involved in the regulation of cytokine production and cell proliferation within tumour and normal tissues (13, 14). It has long been recognized that weight loss (cachexia) is the single most common cause of death in patients with cancer and initial malnutrition indicates a poor prognosis. For a tumour to grow and spread it must induce the formation of new blood vessels and degrade the extracellular matrix. The inflammatory response may have significant roles to play in the above mechanisms, thus contributing to the decline of the host and progression of the tumour. Due to the anti-inflammatory properties of Bifidobacterium longum infantis these bacterial strains they may reduce the rate of malignant cell transformation. Furthermore, intestinal bacteria can produce, from dietary compounds, substances with genotoxic, carcinogenic and tumour-promoting activity and gut bacteria can activate pro-carcinogens to DNA reactive agents (15). In general, species of Bifidobacterium have low activities of xenobiotic metabolizing enzymes compared to other populations within the gut such as bacteroides, eubacteria and clostridia. Therefore, increasing the number of Bifidobacterium bacteria in the gut could beneficially modify the levels of these enzymes.

### Vaccine/Drug Delivery

The majority of pathogenic organisms gain entry via mucosal surfaces. Efficient vaccination of these sites protects against invasion by a particular infectious agent. Oral vaccination strategies have concentrated, to date, on the use of attenuated live pathogenic organisms or purified encapsulated antigens (16). Probiotic bacteria, engineered to produce antigens from an infectious agent, in vivo, may provide an attractive alternative as these bacteria are considered to be safe for human consumption (GRAS status).

Murine studies have demonstrated that consumption of probiotic bacteria expressing foreign antigens can elicit protective immune responses. The gene encoding tetanus toxin fragment C (TTFC) was expressed in Lactococcus lactis and mice were immunized via the oral route. This system was able to induce antibody titers significantly high enough to protect the mice from lethal toxin challenge. In addition to antigen presentation, live bacterial vectors can produce bioactive compounds, such as immunostimulatory cytokines, in vivo. L. lactis secreting bioactive human IL-2 or IL-6 and TTFC induced 10-15 fold higher serum IgG titres in mice immunized intranasally (17). However, with this particular bacterial strain, the total IgA level was not increased by coexpression with these cytokines. Other bacterial strains, such as Streptococcus gordonii, are also being examined for their usefulness as mucosal vaccines. Recombinant S. gordonii colonizing the murine oral and vaginal cavities induced both mucosal and systemic antibody responses to antigens expressed by this bacterial (18). Thus oral immunization using probiotic bacteria as vectors would not only protect the host from infection, but may replace the immunological stimuli that the pathogen would normally elicit thus contributing to the immunological education of the host.

### Prebiotics

The introduction of probiotic organisms is accomplished by the ingestion of the micro-organism in a suitable carrier. It would be advantageous to provide a medium that would promote the growth of these probiotic strains in the large bowel. The addition of one or more oligosaccharides, polysaccharides, or other prebiotics enhances the growth of lactic acid bacteria in the gastrointestinal tract. Prebiotics refers to any non-viable food component that is specifically fermented in the colon by indigenous bacteria thought to be of positive value, e.g. bifidobacteria, lactobacilli. Types of prebiotics may include those that contain fructose, xylose, soya, galactose, glucose and mannose. The combined administration of a probiotic strain with one or more prebiotic compounds may enhance the growth of the administered probiotic in vivo resulting in a more pronounced health benefit, and is termed synbiotic.

### Other Active Ingredients

It will be appreciated that the probiotic strains may be administered prophylactically or as a method of treatment either on its own or with other probiotic and/or prebiotic materials as described above. In addition, the bacteria may be used as part of a prophylactic or treatment regime using other active materials such as those used for treating inflammation or other disorders especially those with an immunological involvement. Such combinations may be administered in a single formulation or as separate formulations administered at the same or different times and using the same or different routes of administration.

The invention is not limited to the embodiments herein before described which may be varied in detail.

### REFERENCES

1. McCracken V. J. and Gaskins H. R. Probiotics and the immune system. In: Probiotics a critical review, Tannock, G W (ed), Horizon Scientific Press, UK. 1999, p. 85-113.
2. Savage D. C. Interaction between the host and its microbes. In: Microbial Ecology of the Gut, Clark and Bauchop (eds), Academic Press, London. 1977, p. 277-310.
3. Kagnoff M. F. Immunology of the intestinal tract. Gastroenterol. 1993; 105 (5): 1275-80.
4. Lamm M. E. Interaction of antigens and antibodies at mucosal surfaces. Ann. Rev. Microbiol. 1997; 51: 311-40.
5. Raychaudhuri S., Rock K L. Fully mobilizing host defense: building better vaccines. Nat biotechnol., 1998; 16: 1025-31.
6. Stallmach A., Strober W, MacDonald T T, Lochs H, Zeitz M. Induction and modulation of gastrointestinal inflammation. Immunol. Today, 1998; 19 (10): 438-41.
7. de Waal Malefyt R, Haanen J, Spits H, Roncarolo M G, te Velde A, Figdor C, Johnson K, Kastelein R, Yssel H, de Vries J E. Interleukin 10 (IL-10) and viral IL-10 strongly reduce antigen-specific human T cell proliferation by diminishing the antigen-presenting capacity of monocytes via downregulation of class II major histocompatibility complex expression. J Exp Med 1991 Oct. 1; 174(4):915-24.
8. Masco L, Huys G, Gevers D, Verbrugghen L, Swings J. Identification of Bifidobacterium species using rep-PCR fingerprinting. Syst Appl Microbiol. 2003 November; 26(4):557-63. PMID: 14666984.
9. Tagg, J R, Dajani, A S, Wannamaker, L W. Bacteriocins of Gram positive bacteria. Bacteriol Rev. 1976; 40: 722-756.
10. Crabbe P. A., H. Bazin, H. Eyssen, and J. F. Heremans. The normal microbial flora as a major stimulus for proliferation of plasma cells synthesizing IgA in the gut. The germ free intestinal tract. Into. Arch. Allergy Appl Immunol, 1968; 34: 362-75.
11. Henderson B., Poole, S and Wilson M. 1998. In “Bacteria-Cytokine interactions in health and disease. Portland Press, 79-130.
12. Arai K I, Lee F, Miyajima A, Miyatake S, Arai N, Yokota T. Cytokines: coordinators of immune and inflammatory responses. Annu Rev Biochem 1990; 59:783-836.
13. McGee D W, Bamberg T, Vitkus S J, McGhee J R. A synergistic relationship between TNF-alpha, IL-1 beta, and TGF-beta 1 on IL-6 secretion by the IEC-6 intestinal epithelial cell line. Immunology 1995 September; 86(1):6-11.
14. Wu S, Meeker W A, Wiener J R, Berchuck A, Bast R C Jr, Boyer C M. Transfection of ovarian cancer cells with tumour necrosis factor alpha (TNF-alpha) antisense mRNA abolishes the proliferative response to interleukin-1 (IL-1) but not TNF-alpha. Gynecol Oncol 1994 April; 53(1):59-63.
15. Rowland I. R. Toxicology of the colon: role of the intestinal microflora. In: Gibson G. R. (ed). Human colonic bacteria: role in nutrition, physiology and pathology, 1995, pp 155-174. Boca Raton CRC Press.
16. Walker, R. I. New strategies for using mucosal vaccination to achieve more effective immunization. Vaccine, 1994; 12: 387-400.
17. Steidler L., K. Robinson, L. Chamberlain, K. M Scholfield, E. Remaut, R. W. F. Le Page and J. M. Wells. Mucosal delivery of murine interleukin-2 (IL-2) and IL-6 by recombinant strains of Lactococcus lactis coexpressing antigen and cytokine. Infect. Immun., 1998; 66:3183-9.
18. Medaglini D., G. Pozzi, T. P. King and V. A. Fischetti. Mucosal and systemic immune responses to a recombinant protein expressed on the surface of the oral commensal bacterium Streptococcus gordonii after oral colonization. Proc. Natl. Acad. Sci. USA, 1995; 92:6868-72 McCracken V. J. and Gaskins H. R, ‘Probiotics a critical review’, Horizon Scientific Press, UK 1999, p. 278.
19. Marson, A., Kretschmer, K., Frampton, G. M., Jacobsen, E. S., Polansky, J. K., MacIsaac, K. D., Levine, S. S., Fraenkel, E., von Boehmer, H. and Young, R. A. Foxp3 occupancy and regulation of key target genes during T-cell stimulation. Letters to Nature, 2007.
20. Kay A B. Allergy and allergic diseases. N Engl J Med 2001; 344:30-7.
21. Strachan D P. Hay fever, hygiene, and household size. BMJ 1989; 299:1259-60.
22. Romagnani S. The increased prevalence of allergy and the hygiene hypothesis: missing immune deviation, reduced immune suppression, or both? Immunol 2004; 112:352-63.
23. Akdis M, Akdis C A. Therapeutic manipulation of immune tolerance in allergic disease. Nat Rev Drug Discov 2009; 8:645-60.
24. O'Mahony L, Feeney M, O'Halloran S, Murphy L, Kiely B, Fitzgibbon J, et al. Probiotic impact on microbial flora, inflammation and tumour development in IL-10 knockout mice. Alim Pharmacol Ther 2001; 15:1219-25.
25. McCarthy J, O'Mahony L, O'Callaghan L, Sheil B, Vaughan E E, Fitzsimons N, et al. Double blind, placebo controlled trial of two probiotic strains in interleukin 10 knockout mice and mechanistic link with cytokine balance. Gut 2003; 52:975-80.
26. O'Mahony L, McCarthy J, Kelly P, Hurley G, Luo F, Chen K, et al. Lactobacillus and bifidobacterium in irritable bowel syndrome: symptom responses and relationship to cytokine profiles. Gastroenterology 2005; 128:541-51.
27. Forsythe P, Inman M D, Bienenstock J. Oral treatment with live lactobacillus reuteri inhibits the allergic airway response in mice. Am J Respir Crit. Care Med 2007; 175:561-9.
28. Kalliomaki M, Salminen S, Poussa T, Arvilommi H, Isolauri E. Probiotics and prevention of atopic disease: 4-year follow-up of a randomised placebo-controlled trial. Lancet 2003; 361:1869-71.
29. Kalliomaki M, Salminen S, Arvilommi H, Kero P, Koskinen P, Isolauri E. Probiotics in primary prevention of atopic disease: A randomised placebo-controlled trial. Lancet 2001; 357:1076-9.
30. Weston S, Halbert A, Richmond P, Prescott S L. Effects of probiotics on atopic dermatitis: A randomised controlled trial. Arch Dis Child 2005; 90:892-7.
31. Helin T, Haahtela S, Haahtela T. No effect of oral treatment with an intestinal bacterial strain, Lactobacillus rhamnosus (ATCC 53103), on birch-pollen allergy: a placebo controlled double-blind study. Allergy 2002:57; 243-6.
32. Karim M, Kingsley C I, Bushell A R, Sawitzki B S, Wood K J. Alloantigen-induced CD25+CD4+ regulatory T cells can develop in vivo from CD25−CD4+ precursors in a thymus-independent process. J Immunol 2004; 172:923-8.
33. Chen W, Jin W, Hardegen N, Lei K J, Li L, et al. Conversion of peripheral CD4+CD25− naive T cells to CD4+CD25+ regulatory T cells by TGF-□ induction of transcription factor Foxp3. J Exp Med 2003; 198:1875-86.
34. Sun C M, Hall J A, Blank R B, Bouladoux N, Oukka M, et al. Small intestine lamina propria dendritic cells promote de novo generation of Foxp3 T reg cells via retinoic acid. J Exp Med 2007; 204: 1775-85.
35. Coombes J L, Siddiqui K R, Arancibia-Cárcamo C V, Hall J, Sun C M, et al. A functionally specialized population of mucosal CD103+ DCs induces Foxp3+ regulatory T cells via a TGF-beta and retinoic acid-dependent mechanism. J Exp Med 2007; 204:1757-64.
36. Di Giacinto C, Marinaro M, Sanchez M, Strober W, Boirivant M. Probiotics Ameliorate Recurrent Th1-Mediated Murine Colitis by Inducing IL-10 and IL-10-Dependent TGF-1 Bearing Regulatory Cells. J Immunol 2005; 174:3237-46.
37. O'Mahony C, Scully P, O'Mahony D, Murphy S, Lyons A, MacSharry J, et al. Commensal-Induced Regulatory T Cells Mediate Protection against Pathogen-Stimulated NF-κB Activation. PLOS Pathogens 2008; 4:e1000112.
38. Karimi K, Inman M D, Bienenstock J, Forsythe P. Lactobacillus reuteri-induced regulatory T cells protect against an allergic airway response in mice. Am J Respir Crit. Care Med. 2009; 179:186-93.
39. Murphy C, Murphy S, O'Brien F, O'Donoghue M, Boileau T, Sunvold G, et al. Metabolic activity of probiotics-Oxalate degradation. Vet Microbiol 2009; 136:100-7.
40. Nagaishi T, Chen Z, Chen L, Iijima H, Nakajima A, Blumberg R S. CEACAM1 and the regulation of mucosal inflammation. Mucosal Immunol 2008; 1 Suppl 1:S39-42.
41. Brigelius-Flohé R. Glutathione peroxidases and redox-regulated transcription factors. Biol Chem 2006; 387:1329-35.
42. Yamamoto M, Takeda K. Role of nuclear IkappaB proteins in the regulation of host immune responses. J Infect Chemother 2008; 14:265-9.
43. Kojima R, Randall J D, Ito E, Manshio H, Suzuki Y, Gullans S R. Regulation of expression of the stress response gene, Osp94: identification of the tonicity response element and intracellular signalling pathways. Biochem J 2004; 380:783-94.
44. Mukherjee S, Partch C L, Lehotzky R E, Whitham C V, Chu H, Bevins C L, et al. Regulation of C-type lectin antimicrobial activity by a flexible N-terminal prosegment. J Biol Chem 2009; 284:4881-8.
45. Miyanaka Y, Ueno Y, Tanaka S, Yoshioka K, Hatakeyama T, Shimamoto M, et al. Clinical significance of mucosal suppressors of cytokine signaling 3 expression in ulcerative colitis. World J Gastroenterol 2007; 13:2939-44.
46. Kanamori M, Suzuki H, Saito R, Muramatsu M, Hayashizaki Y. T2BP, a novel TRAF2 binding protein, can activate NF-kappaB and AP-1 without TNF stimulation. Biochem Biophys Res Commun 2002; 290:1108-13.
47. Straus D S, Glass C K. Anti-inflammatory actions of PPAR ligands: new insights on cellular and molecular mechanisms. Trends Immunol 2007; 28:551-8.
48. Matsuzaki T, Yamazaki R, Hashimoto S, Yokokura T. The effect of oral feeding of Lactobacillus casei strain Shirota on immunoglobulin E production in mice. J Dairy Sci 1998; 81:48-53.
49. Zuany-Amorim C, Sawicka E, Manlius C, Le Moine A, Brunet L R, Kemeny D M, et al. Suppression of airway eosinophilia by killed mycobacterium vaccae induced allergen-specific regulatory T-cells. Nat Med 2002; 8:625-9.
50. Leech M D, Benson R A, De Vries A, Fitch P M, Howie S E. Resolution of Der p1-induced allergic airway inflammation is dependent on CD4+CD25+Foxp3+ regulatory cells. J Immunol 2007; 179:7050-8.
51. McGlade J P, Gorman S, Zosky G R, Larcombe A N, Sly P D, Finlay-Jones J J, et al. Suppression of the asthmatic phenotype by ultraviolet b-induced, antigen-specific regulatory cells. Clin Exp Allergy 2007; 37:1267-76.
52. Strickland D H, Stumbles P A, Zosky G R, Subrata L S, Thomas J A, Turner D J, et al. Reversal of airway hyperresponsiveness by induction of airway mucosal CD4+CD25+ regulatory Tcells. J Exp Med 2006; 203:2649-60.
53. Wu K, Bi Y, Sun K, Xia J, Wang Y, Wang C. Suppression of allergic inflammation by allergen-DNA-modified dendritic cells depends on the induction of Foxp3+ regulatory T cells. Scand J Immunol 2008; 67:140-51.
54. Boudousquié C, Pellaton C, Barbier N, Spertini F. CD4+CD25+ T cell depletion impairs tolerance induction in a murine model of asthma. Clin Exp Allergy 2009; 39:1415-26.
55. Larché M, Akdis C A, Valenta R. Immunological mechanisms of allergen-specific immunotherapy. Nat Rev Immunol 2006; 6:761-71.

1. A Bifidobacterium longum strain AH1206 having the accession number NCIMB 41382 or mutants or variants thereof.

2. Bifidobacterium strain as claimed in claim 1 wherein the mutant is a genetically modified mutant, or a naturally occurring variant of Bifidobacterium.

3. (canceled)

4. (canceled)

5. The Bifidobacterium strain as claimed in claim 1 wherein the strain is in the form of a biologically pure culture.

6. An isolated strain of Bifidobacterium NCIMB 41382.

7. The Bifidobacterium strain as claimed in claim 1 in the form of viable cells or in the form of non-viable cells.

8. (canceled)

9. (canceled)

10. (canceled)

11. The formulation which comprises a Bifidobacterium strain as claimed in claim 1.

12. The formulation as claimed in claim 11 which further comprises one or more selected from the group comprising another probiotic material, a prebiotic material, and an ingestable carrier.

13. (canceled)

14. (canceled)

15. The formulation as claimed in claim 14 wherein the ingestable carrier is a pharmaceutically acceptable carrier such as a capsule, tablet or powder, or a food product such as acidified milk, yoghurt, frozen yoghurt, milk powder, milk concentrate, cheese spreads, dressings or beverages.

16. (canceled)

17. The formulation as claimed in claim 11 which further comprises one or more selected from the group comprising a protein and/or peptide, in particular proteins and/or peptides that are rich in glutamine/glutamate, a lipid, a carbohydrate, a vitamin, mineral trace element, an adjuvant, a bacterial component, a drug entity and/or a biological compound.

18. The formulation as claimed in claim 11 wherein the Bifidobacterium strain is present in an amount of more than 10⁶ cfu per gram of the formulation.

19. (canceled)

20. (canceled)

21. (canceled)

22. (canceled)

23. (canceled)

24. The food stuff comprising Bifidobacterium strain as claimed in claim 1.

25. The medicament comprising a Bifidobacterium strain as claimed in claim 1.

26. The method for the prophylaxis and/or treatment of undesirable inflammatory activity such as the prophylaxis and/or treatment of undesirable gastrointestinal inflammatory activity such as inflammatory bowel disease eg. Crohns disease or ulcerative colitis, irritable bowel syndrome; pouchitis; or post infection colitis comprising the step of administering a Bifidobacterium strain as claimed in claim 1.

27. (canceled)

28. The method for the prophylaxis and/or treatment of gastrointestinal cancer(s), or the prophylaxis and/or treatment of systemic disease such as rheumatoid arthritis, or the prophylaxis and/or treatment of autoimmune disorders due to undesirable inflammatory activity or the prophylaxis and/or treatment of cancer due to undesirable inflammatory activity comprising the step of administering a Bifidobacterium strain as claimed in claim 1.

29. (canceled)

30. (canceled)

31. (canceled)

32. (canceled)

33. The method for the prophylaxis and/or treatment of diarrhoeal disease due to undesirable inflammatory activity, such as Clostridium difficile associated diarrhoea, Rotavirus associated diarrhoea or post infective diarrhoea or diarrhoeal disease due to an infectious agent, such as E. coli comprising the step of administering a Bifidobacterium strain as claimed in claim 1.

34. An anti-inflammatory biotherapeutic agent for the prophylaxis and/or treatment of undesirable inflammatory activity comprising a Bifidobacterium strain as claimed in claim 1.

35. (canceled)

36. The method for the prophylaxis and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising the step of administering a Bifidobacterium strain as claimed in claim 1 or an active derivative fragment or mutant thereof.

37. (canceled)

38. The anti-inflammatory biotherapeutic agent as claimed in claim 34 wherein the anti-inflammatory biotherapeutic agent reduces the level(s) of pro inflammatory cytokines, and/or reduces levels of IgE.

39. (canceled)

40. The medicament for treating asthma and/or allergy comprising a Bifidobacterium strain as claimed in claim 1.

41. The medicament as claimed in claim 40 wherein the medicament is in a form suitable for inhalation.

42. (canceled)


SEQUENCE LISTING (5 sequences)

SEQ ID NO: 1 - DNA, 488 bp, Bifidobacterium longum
  misc_feature (4)..(4): n is a, c, g, or t
tgcngncacacgtcaccacacggtgtcgcatggccgcaaggnatccttcctagcaaattcccagnacgacaaatcatcacactaaaatgatcacaaaacgatcgaaacaaacactaaaaatagagttngattngaaatnattngaaatnaacagcnagaacgaggaatnaaaggnaaccccgtnttgnttgngtncactatncagttttnaagccaccacgcaccancacgccgtncggacgggaccagcccgncatnaggnacgatgggcatngaatcgcgccnggncaaancctggggtggcgatncgggagcccaaaagcgcatncacaccactnccgcggaacattccacgacggacgcnccgnaagnccatgatntttttcacaccagcagccccaagncgccgcgactgncgcgacgccngggctcgcaccgncggacgaacatncggccgtattntncgtanaaaggaggtatcccancaa

SEQ ID NO: 2 - DNA, 476 bp, Bifidobacterium longum
  misc_feature (3)..(3): n is a, c, g, or t
agntaagccgaattctccgcggtgcgngccccggcgtcgcggcagtcgcggcggcctggggctgctgntgtggaagagatcatgggctttcggtgcgtccgtcgtgggatgttccgcgggagtggtgtggatgcgcttttgggctcccggatcgccaccccaggctttggcctggcgcgattcgatgcccatcgtgcctgatggcgggctggtcccgtccggacggcgtggtggtgcgtggtggcttgagaactggatagtggacgcgagcaagacngggtttcctttgattcctcgttcttgctgttgatttcgaatcgaactctatttttaatgnttgtttcnancgttttgtgancattttaatgtganganttgtcntctgggaatttgctaagaanganccttgcngccatgcccaccgtgtggtgcntgttgcctgcaagggcntanggtggaagccttgccacccagaa

SEQ ID NO: 3 - DNA, 18 bp, Artificial Sequence
  primer
gctggatcacctcctttc

SEQ ID NO: 4 - DNA, 18 bp, Artificial Sequence
  primer
ctggtgccaaggcatcca

SEQ ID NO: 5 - DNA, 22 bp, Artificial Sequence
  primer
ctacggcaaggcgacgctgacg